(19) United States
(12) Patent Application Publication
(10) Pub. No.: US 2026/0248834 A1
(43) Pub. Date: Aug. 27, 2026

(54) PHARMACEUTICAL COMPOSITION AND METHOD FOR TREATMENT OF HUMAN IMMUNODEFICIENCY VIRUS INFECTIONS

(71) Applicant: Brii Biosciences, Inc., Durham, NC (US)

(72) Inventors: Zhi HONG, Durham, NC (US); Michael Zhiyan WANG, Foster City, CA (US); Lianhong XU, Palo Alto, CA (US)

(21) Appl. No.: 18/854,478

(22) PCT Filed: Apr. 5, 2023
(86) PCT No.: PCT/US2023/065355

§ 371 Date: Oct. 4, 2024

Related U.S. Application Data
(60) Provisional application No. 63/327,839, filed on Apr. 6, 2022.

(51) Int. Cl. A61K 31/7076; A61K 9/00; A61K 31/34; A61K 31/4418; A61K 31/4439; A61K 31/4985; A61K 31/505; A61K 31/536; A61K 31/5365; A61K 31/537; A61K 31/5377; A61K 31/675

(52) U.S. Cl. CPC A61K 31/7076; A61K 9/0024; A61K 9/0053; A61K 31/34; A61K 31/4418; A61K 31/4439; A61K 31/4985; A61K 31/505; A61K 31/536; A61K 31/5365; A61K 31/537; A61K 31/5377; A61K 31/675

(57) ABSTRACT

This disclosure is directed to pharmaceutical compositions and methods for treating or preventing HIV infection in a subject in need thereof. The composition can comprise an adenosine derivative that can be administered via injection, oral intake, implant, or a combination thereof.

### CROSS-REFERENCE TO RELATED APPLICATIONS

This application claims the benefit of U.S. Provisional Application No. 63/327,839, filed Apr. 6, 2022, which is incorporated herein by reference in its entirety.

### FIELD OF THE DISCLOSURE

This disclosure is directed to pharmaceutical compositions comprising an adenosine derivative and methods for using the adenosine derivative to treat or prevent acquired immunodeficiency syndrome (AIDS), human immunodeficiency virus infection (HIV), (e.g., HIV-1, HIV-2, multidrug resistant HIV infection) or a combination thereof.

### BACKGROUND

Retroviruses such as human immunodeficiency virus (HIV) has been linked to the immunosuppressive disease known as acquired immunodeficiency syndrome (AIDS). Multiple strains of retrovirus, such as HIV type-1 (HIV-1) and type-2 (HIV-2) are known to be related to the diseases. The HIV retrovirus infected individuals can be initially asymptomatic, but then develop AIDS related complex (ARC) followed by AIDS. Replication of HIV by a host cell requires integration of the viral genome into the DNA of host cells. A key step in the process involves transcription of the viral RNA genome into DNA via an enzyme known as reverse transcriptase (RT).

A reverse transcriptase typically can have multiple enzymatic functions that can act (1) as an RNA-dependent DNA polymerase transcribing a single-stranded DNA copy of the viral RNA (first DNA), (2) as a ribonuclease destroying the original viral RNA and frees the DNA just produced from the original RNA, and (3) as a DNA-dependent DNA polymerase producing a second, complementary DNA strand using the first DNA strand as a template. The two DNA strands then form double-stranded DNA, which is integrated into the genome of the host cells by an integrase enzyme.

A number of compounds can inhibit reverse transcriptase (RT) activity. These compounds can be useful for the treatment of HIV infection in humans by inhibiting HIV replication in infected cells or individuals. Examples of the compounds approved for use in treating HIV infection and AIDS include nucleoside RT inhibitors (NRTI) such as 3′-azido-3′-deoxythymidine (AZT, also known as Zidovudine (ZDV), azidothymidine (AZT)), 2′,3′-dideoxyinosine (ddl), 2′,3′-dideoxycytidine (ddC), d4T, 3TC, abacavir, emtricitabine, and tenofovir disoproxil fumarate, as well as non-nucleoside RT inhibitors (NNRTI) such as nevirapine, delavirdine, efavirenz, rilpivirine and doravirine (DHHS guidelines: https://aidsinfo.nih.gov/understanding-hiv-aids, Iyidogan & Anderson, Viruses, 6, 4095-4139, 2014, doi:10.3390/v6104095; Hayakawa et al., Antiviral Chem & Chemotherapy, 15:169-187, 2004; Ohrul et al., J. Med. Chem. 43, 4516-4525, 2000; Pauwels, Antiviral Research, 71, 77-89, 2006).

An adenosine derivative EFdA (4′-ethynyl-2-fluoro-2′-deoxyadenosine, also known as MK-8591 or islatravir) is a long-acting (LA) NRTTI that has been demonstrated to have anti-HIV activity via inhibiting reverse transcriptase by preventing translocation (U.S. Pat. Nos. 7,339,053, 7,625,877, 8,039,614. Singh et al., Pharmaceuticals, 12, 62, 2019, DOI: 10.3390/ph12020062, each of which is incorporated by reference herein in its entirety). This compound has broad inhibitory activity and potency for different subtypes and mutations including HIV-1, HIV-2, and multidrug resistant (MDR) and wildtype (WT) strains, and reverse transcriptase inhibitor (RTI) resistant viruses. Some modified EFdA analogs and prodrugs have been described in U.S. Patent Publication No.: 2018/0002366, incorporated by reference herein in its entirety.

A common issue that arises from the treatment of HIV infection with anti-retroviral inhibitory compounds is resistance of the viruses to the inhibitors. Mutations that occur in the reverse transcriptase segment of the pol gene often resulted in resistance to current available NRTIs and/or NNRTIs. Therefore, there is a continuing need for new RT inhibitors that are effective against HIV strains including mutant HIV and multidrug-resistant HIV strains.

Another issue is the side effect and toxicity of the treatment drug. Recently, it has been reported that decrease in total lymphocyte and CD4+ T-cell counts occurred in some subjects who received islatravir in clinical trials indicating potential undesired side effects.

Yet another common issue is the medication adherence that is essential for individuals with HIV infections to have successful therapy over a lifetime. Adherence to a daily regimen can be challenging, which also has negative impact on the patient's quality of life with daily reminder of their HIV status. Increasing patient adherence to a drug regimen can potentially be achieved through reducing the dosing frequency. Therefore, there is a need to identify long-acting compounds or regimens (for example, once a week, once a month or once every half year therapy) for patients to overcome these challenges tied to taking daily, oral medications.

Therefore, there is a continuing need for new drugs and treatment methods that are effective against HIV infections including mutant HIV, less side effect and toxicity, and better medication adherence.

### SUMMARY

The present disclosure is directed to methods of treating an HIV infection, comprising administering to a subject in need thereof an adenosine derivative, or a pharmaceutically acceptable salt, tautomer, or solvate thereof, wherein the subject is administered a dose of the adenosine derivative in an amount from about 0.1 mg to about 25 mg.

In some embodiments, the adenosine derivative is a compound having the structure:

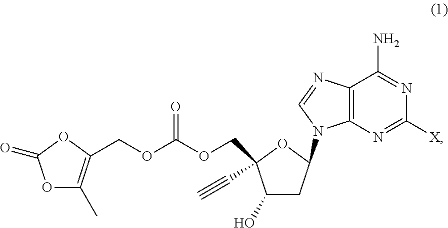

or a pharmaceutically acceptable salt, tautomer, or solvate thereof, wherein X is halogen.

In some embodiments, the adenosine derivative is a compound having the structure:

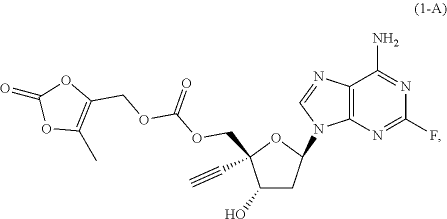 [SMILES: C#C[C@]1(COC(=O)OCc2oc(=O)oc2C)O[C@@H](n2cnc3c(N)nc(F)nc32)C[C@@H]1O]

or a pharmaceutically acceptable salt, tautomer, or solvate thereof.

The present disclosure is further directed to methods of treating an HIV infection, comprising administering to a subject in need thereof an adenosine derivative having the structure:

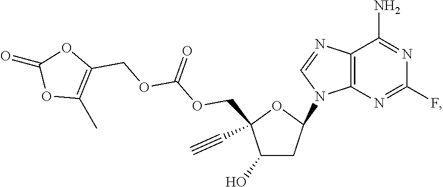 [SMILES: C#C[C@]1(COC(=O)OCc2oc(=O)oc2C)O[C@@H](n2cnc3c(N)nc(F)nc32)C[C@@H]1O]

wherein the subject is administered a tablet comprising a 0.25 mg or 0.38 mg dose of the adenosine derivative.

In some embodiments, the subject is administered a once-weekly or once-daily dose of the adenosine derivative. In some embodiments, the subject is administered a once-weekly dose of the adenosine derivative. In some embodiments, the subject is administered a once-daily dose of the adenosine derivative.

In some embodiments, provided herein are methods of treating an HIV infection in a subject comprising orally administering to the subject a once-weekly dose of from about 0.1 mg to about 25 mg of an adenosine derivative or a pharmaceutically acceptable salt thereof for a plurality of weeks.

In some embodiments, the adenosine derivative has the structure:

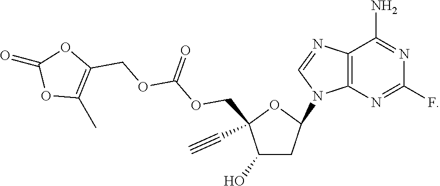 [SMILES: C#C[C@]1(COC(=O)OCc2oc(=O)oc2C)O[C@@H](n2cnc3c(N)nc(F)nc32)C[C@@H]1O]

In some embodiments, the adenosine derivative is EFdA (4′-ethynyl-2-fluoro-2′-deoxyadenosine).

In some embodiments, the once-weekly dose comprises from about 1 mg to about 10 mg of the adenosine derivative. In some embodiments, the once-weekly dose comprises from about 1 mg to about 2 mg of the adenosine derivative. In some embodiments, the once-weekly dose comprises from about 1.3 mg to about 1.75 mg of the adenosine derivative. In some embodiments, the once-weekly dose comprises about 1.5 mg of the adenosine derivative. In some embodiments, the once-weekly dose comprises about 1.25 mg of the adenosine derivative. In some embodiments, the once-weekly dose comprises about 2.3 mg of the adenosine derivative. In some embodiments, the once-weekly dose comprises about 1 mg to about 8 mg of the adenosine derivative. In some embodiments, the once-weekly dose comprises 2±0.5 mg of the adenosine derivative. In some embodiments, the once-weekly dose comprises 3±0.5 mg of the adenosine derivative. In some embodiments, the once-weekly dose comprises 4±0.5 mg of the adenosine derivative. In some embodiments, the once-weekly dose comprises 5±0.5 mg of the adenosine derivative. In some embodiments, the once-weekly dose comprises 6±0.5 mg of the adenosine derivative.

In some embodiments, the once-weekly dose is administered as a tablet comprising the adenosine derivative or pharmaceutically acceptable salt thereof.

In some embodiments, provided herein are methods of treating an HIV infection in a subject comprising orally administering to the subject a once-daily dose of from about 0.1 mg to about 10 mg of an adenosine derivative or a pharmaceutically acceptable salt thereof for a plurality of weeks.

In some embodiments, the adenosine derivative has the structure:

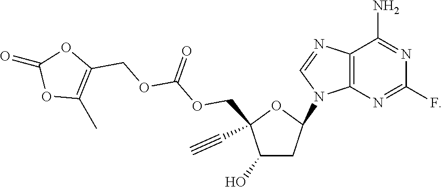 [SMILES: C#C[C@]1(COC(=O)OCc2oc(=O)oc2C)O[C@@H](n2cnc3c(N)nc(F)nc32)C[C@@H]1O]

In some embodiments, the adenosine derivative is EFdA (4′-ethynyl-2-fluoro-2′-deoxyadenosine).

In some embodiments, the once-daily dose comprises from about 0.1 mg to about 5 mg of the adenosine derivative. In some embodiments, the once-daily dose comprises from about 0.1 mg to about 0.75 mg of the adenosine derivative. In some embodiments, the once-daily dose comprises about 0.25 mg or about 0.38 mg of the adenosine derivative. In some embodiments, the once-daily dose comprises about 0.25 mg of the adenosine derivative. In some embodiments, the once-daily dose comprises about 0.38 mg of the adenosine derivative.

In some embodiments, the once-daily dose is administered as a tablet comprising the adenosine derivative or pharmaceutically acceptable salt thereof.

In some embodiments of the methods provided herein, the HIV infection is caused by wild-type HIV-1, NRTI-resistant HIV-1, HIV-2, HIV having a M184V mutation, HIV having K65R, or multidrug resistant HIV. In some embodiments, the HIV infection is an HIV-1 infection.

In some embodiments of the methods provided herein, the subject has reduced expression of: (a) deoxycytidine kinase; (b) adenosine deaminase; and/or (c) purine nucleoside phosphorylase (PNP) compared to a treatment-naïve subject. In some embodiments, the subject is at risk for CD4+ lymphocyte count reduction compared to a treatment-naïve subject.

In some embodiments, a method as provided herein further comprises administering to the subject one or more anti-HIV agents selected from atazanavir, atazanavir sulfate, bictegravir, cabotegravir, dolutegravir, doravirine, efavirenz, tenofovir disoproxil fumarate, tenofovir alafenamide, elvitegravir, etravirine, darunavir, a combination of darunavir and cobicistat, rilpivirine, lenacapavir, and a combination thereof.

The present disclosure is further directed to pharmaceutical compositions comprising an adenosine derivative and one or more pharmaceutically acceptable carriers, wherein the pharmaceutical composition comprises from about 0.1 mg to 25 mg of the adenosine derivative.

In some embodiments, the adenosine derivative is a compound having the structure:

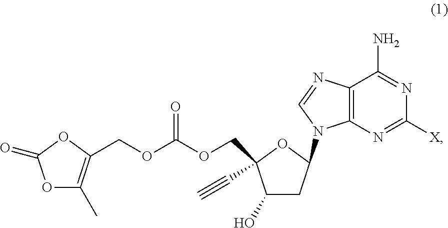

or a pharmaceutically acceptable salt, tautomer, or solvate thereof, wherein X is a halogen.

In some embodiments, X is F or Cl. In some embodiments, X is F. In some embodiments, X is Cl.

In some embodiments, the adenosine derivative is a compound having the structure:

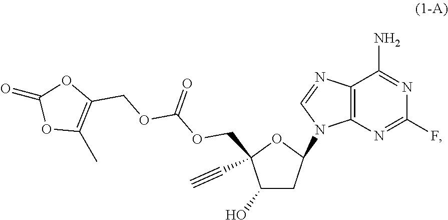 [SMILES: C#C[C@]1(COC(=O)OCc2oc(=O)oc2C)O[C@@H](n2cnc3c(N)nc(F)nc32)C[C@@H]1O]

or a pharmaceutically acceptable salt, tautomer, or solvate thereof.

In some embodiments, the adenosine derivative is:

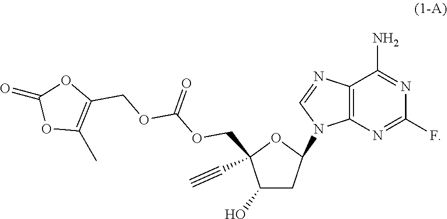 [SMILES: C#C[C@]1(COC(=O)OCc2oc(=O)oc2C)O[C@@H](n2cnc3c(N)nc(F)nc32)C[C@@H]1O]

In some embodiments, the pharmaceutical composition comprises from about 1 mg to about 10 mg of the adenosine derivative. In some embodiments, the pharmaceutical composition comprises from about 1.3 mg to about 1.75 mg of the adenosine derivative. In some embodiments, the pharmaceutical composition comprises about 1.5 mg of the adenosine derivative. In some embodiments, the pharmaceutical composition comprises from about 0.1 mg to about 0.75 mg of the adenosine derivative. In some embodiments, the pharmaceutical composition comprises about 0.25 mg or about 0.38 mg of the adenosine derivative. In some embodiments, the pharmaceutical composition comprises about 0.25 mg of the adenosine derivative. In some embodiments, the pharmaceutical composition comprises about 0.38 mg of the adenosine derivative.

The present disclosure is further directed to process for diagnosis and treatment or prevention of HIV infection in a subject in need thereof. The process can comprise measuring purine nucleoside phosphorylase (PNP) activity from a sample of a subject prior to administering a pharmaceutical composition.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Following are more detailed descriptions of various concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

As used herein, the term “halogen” (or “halo”) refers to fluorine, chlorine, bromine and iodine (alternatively referred to as fluoro (—F), chloro (—Cl), bromo (—Br), and iodo (—I)).

As used herein, the term “isomer” refers to a structural isomer, such as a group or an atom positioned at different locations of a molecule; stereoisomer, such as chiral isomers, enantiomers, diastereomers and cis/trans isomers; a tautomer, such as amino isomer, imino isomer, or a combination thereof. In non-limiting examples, an adenosine derivative of the present disclosure can have an amino isomer, an imino isomer or a combination thereof. In another non-limiting example, in instances where an —OH substituent is permitted on a heteroaromatic ring and keto-enol tautomerism is possible, it is understood that the substituent might in fact be present, in whole or in part, in the oxo (═O) form. A mixture of isomers can also be suitable. A mixture of isomers can comprise the respective isomers in all ratios. A salt of an isomer can also be suitable. An adenosine derivative of the present disclosure can comprise isomers thereof, one or more salts thereof, one or more solvates including hydrates thereof, solvated salts thereof or a mixture thereof. Absolute stereochemistry or isomer configuration may be determined by X-ray crystallography, by Vibrational Circular Dichroism (VCD) spectroscopy analysis or a combination thereof.

The adenosine derivatives can be identified by names based on the nomenclature recommended by International Union of Pure and Applied Chemistry (IUPAC) or based on nucleosides (Nucleoside-based nomenclature). The adenosine derivatives can also be identified by chemical structure drawings. Unless expressly stated to the contrary in a particular context, the names and the structures may be used interchangeably.

Any of the atoms in a compound disclosed herein may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds disclosed herein.

The compounds can be administered in the form of pharmaceutically acceptable salts or solvates. The term “pharmaceutically acceptable salt” refers to a salt or a solvate which is not biologically or otherwise undesirable (e.g., is neither toxic nor otherwise deleterious to the recipient or subject thereof). A mixture of a compound disclosed herein and one or more salts or solvates thereof is also contemplated herein. Illustrative examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methanesulfonates, propanesulfonates, naphthalene-1-sulfonates, naphthalene-2-sulfonates, and mandelates.

Furthermore, compounds disclosed herein can exist in amorphous form and/or one or more crystalline forms, or a combination thereof.

The term “RNA virus infection” refers to a disease caused by an RNA virus, such as the common cold, influenza, SARS, COVID-19, hepatitis C, hepatitis E, West Nile fever, Ebola virus disease, rabies, polio, and measles.

The term “HIV infection” refers to a disease caused by the human immunodeficiency virus (HIV), such as HIV-1 and HIV-2. In some cases, the HIV infection can be caused by wild-type HIV-1, NRTI-resistant HIV-1, HIV-2, HIV having M184V mutations, HIV having K65R, or multidrug resistant HIV. The term “AIDS” refers to acquired immunodeficiency syndrome, which is caused by HIV infection and an advanced form of the disease.

The term “adenosine derivative” refers to a compound obtained, formed, or imagined to arise from adenosine by the replacement and/or addition of one or more atoms. In some embodiments, the adenosine derivative is obtained from adenosine by a chemical or biological reaction. In some embodiments, the adenosine derivative disclosed herein is a prodrug. In some embodiments, an adenosine derivative A (e.g., an adenosine derivative of Formula (1) as described herein) is a prodrug of adenosine derivative B (e.g. EFdA).

The term “prodrug” refers to a compound that may be converted under physiological conditions or by solvolysis to a biologically active compound described herein. Thus, the term “prodrug” refers to a precursor of a biologically active compound that can be pharmaceutically acceptable. A prodrug may be a biologically inactive or substantially inactive compound which can be metabolized in the body, i.e., in vivo, to produce a drug having a desired activity. The term “substantially inactive” means that a prodrug can have about 1% to about 10% of the activity of the corresponding drug or after being metabolized in vivo, percentage based on weight of the prodrug. In some embodiments, the term “substantially inactive” means that a prodrug has less than about 5% of the activity of the corresponding drug or after being metabolized in vivo, percentage based on weight of the prodrug. The doses for a prodrug and its biologically active compound can be considered to be dose-equivalent when they are the same molar amount. For example, a dose of the adenosine derivative of Formula (1) and its biologically active compound can be considered to dose-equivalent when they are the same molar amount.

The term “anti-HIV agent”, “anti-viral agent” or a grammatical variant refers to a compound, a mixture of one or more compounds, a formulation, a chemical agent or a biological agent such as antibody, protein, peptides, nucleotide, other biological compound, or a combination thereof, that can be directly or indirectly effective in the inhibition of HIV, the treatment or prophylaxis of HIV infection, and/or the treatment, prophylaxis or delay in the onset or progression of AIDS and/or diseases or conditions arising therefrom or associated therewith, an RNA virus infection, or a combination thereof. The anti-HIV agents can comprise HIV antiviral agents, immunomodulators, anti-infectives, vaccines or a combination thereof useful for treating HIV infection or AIDS. Examples of antiviral agents for Treating HIV infection or AIDS include, but are not limited to, under respective trademarks or registered trademarks with respective owners, atazanavir (Reyataz®), darunavir (Prezista®), dolutegravir (Tivicay®), doravirine, efavirenz (EFV, Sustiva®, Stocrin®), cabotegravir, bictegravir, emtricitabine (FTC, Emtriva®), rilpivirine (Edurant®), tenofovir hexadecyloxypropyl (CMX-157), tenofovir alafenamide fumarate, MK-8507, and lenacapavir. Some of the anti-HIV agents shown above can be used in a salt form; for example, atazanavir sulfate, tenofovir alafenamide fumarate or other salts. An anti-HIV agent can have one or more activities such as entry inhibitor (EI); a capsid inhibitor (CAI), fusion inhibitor (FI); integrase inhibitor (InI); protease inhibitor (PI); nucleoside reverse transcriptase inhibitor (nRTI or NRTI) or non-nucleoside reverse transcriptase inhibitor (nnRTI or NNRTI). An anti-HIV agent can comprise two or more agents disclosed herein. The adenosine derivative of the present disclosure can be an anti-HIV agent along or in combination with other anti-HIV agent or agents.

Unless expressly stated to the contrary, all ranges cited herein are inclusive. It is to be understood that any range cited herein includes within its scope all of the sub-ranges within that range. For example, a dosage in a range of from “0.1 to 5 mg” means the dosage of 0.1 mg, 0.2 mg, 0.3 mg, 0.4 mg, 0.5 mg, 0.6 mg, 0.7 mg, 0.8 mg, 0.9 mg, 1.0 mg, 1.1 mg and so on, and 5 mg including all dosages within the range; a dosage in a range of from “0.1 to 10 mg” means the dosage of 0.1 mg, 0.11 mg, 0.2 mg, 0.21 mg, 0.3 mg, 0.4 mg, 0.45 mg, 0.5 mg, 0.6 mg, 0.7 mg, 0.8 mg, 0.9 mg, 1.0 mg, 1.1 mg and so on, and 10 mg including all dosages within the range; a dosage in a range of from “10 to 1000 mg” means the dosage of 10 mg, 10.1 mg, 10.01 mg, 100 mg, 101 mg, 101.1 mg, 101.01 mg and so on, and 1000 mg including all dosages within the range. In another non-limiting example, a time range of “1 to 8 days” means 1 day, 2, days, 3 days, 4 days, 5 days, 6 days, 7 days, and 8 days including all times subranges or each and every individual time point or time points within the range. In another non-limiting example, a time range of “once-daily (QD)” to “once per week (Q1W)” means once every day, once every two days, once every three days, once every 4 days, once every 5 days, once every 6 day and once every 7 days (week) (Q1W). In yet another non-limiting example, a time range of “once per week (Q1W) to once every 8 weeks (Q8W)” means once per week (Q1W), once every two weeks (Q2W), once every 3 weeks (Q3W), once every 4 weeks (Q4W), once every 5 weeks (Q5W), once every 6 weeks (Q6W), once every 7 weeks (Q7W) and once every 8 weeks (Q8W). In yet another non-limiting example, a time range of “once per month (Q1M) to once every 12 months (Q12M)” means once per month (Q1M), once every two months (Q2M), once every 3 months (Q3M), once every 4 months (Q4M), once every 5 months (Q5M), once every 6 months (Q6M), once every 7 months (Q7M), once every 8 months (Q8M), once every 9 months (Q9M), once every 10 months (Q10M), once every 11 months (Q11M), and once every 12 months (Q12M).

Unless otherwise indicated, open terms for example “contain,” “containing,” “include,” “including,” and the like mean “comprising”.

The singular forms “a”, “an”, and “the” are used herein to include plural references unless the context clearly dictates otherwise. Accordingly, unless the contrary is indicated, the numerical parameters set forth in this application are approximations that may vary depending upon the desired properties sought to be obtained by the present disclosure.

The term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11.

The term “injection” refers to intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Other non-parenteral route can include, such as a topical, epidermal or mucosal route of administration, e.g., intranasally, orally, vaginally, rectally, sublingually or topically. The pharmaceutical composition can be in the form of sterile aqueous solutions or dispersions. The pharmaceutical composition can also be formulated in a microemulsion, liposome, or other ordered structure suitable to high drug concentration.

As used throughout this disclosure, the term “adenosine derivative has a formula” or “adenosine derivative comprises a formula” means that an adenosine derivative can have a compound with the formula or identified with the formula or an adenosine derivative can comprise compound with the formula or identified with the formula.

### Methods

In some embodiments, the present disclosure provides methods of treating or preventing HIV infection, comprising administering to a subject in need thereof an adenosine derivative or a pharmaceutically acceptable salt, tautomer, or solvate thereof, wherein the subject is administered a dose of the adenosine derivative in an amount from about 0.1 mg to about 25 mg.

In some embodiments, the present disclosure provides methods of treating an HIV infection in a subject comprising orally administering to the subject a once-weekly dose of from about 0.1 mg to about 25 mg of an adenosine derivative or a pharmaceutically acceptable salt thereof for a plurality of weeks.

In some embodiments, the present disclosure provides methods of treating HIV infection in a subject comprising orally administering to the subject a once-daily dose of from about 0.1 mg to about 5 mg of an adenosine derivative or a pharmaceutically acceptable salt thereof for a plurality of weeks.

The methods of preventing HIV infection disclosed herein can refer to pre-exposure prophylaxis (PrEP) of HIV infection in an uninfected subject or post-exposure prophylaxis (PEP) of HIV infection in an uninfected subject. In some embodiments, the methods of preventing HIV infection refer to HIV PrEP.

In some embodiments, the adenosine derivative is a compound having the structure:

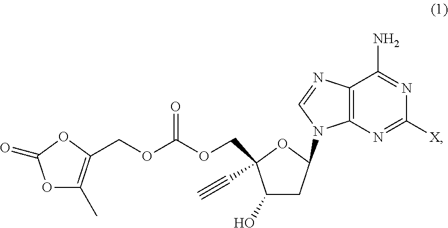

or a pharmaceutically acceptable salt, tautomer, or solvate thereof, wherein X is halogen.

In some embodiments, X is F or Cl. In some embodiments, X is F. In some embodiments, X is Cl.

In some embodiments, the adenosine derivative is a compound having the structure:

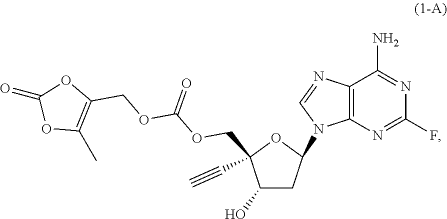 [SMILES: C#C[C@]1(COC(=O)OCc2oc(=O)oc2C)O[C@@H](n2cnc3c(N)nc(F)nc32)C[C@@H]1O]

or a pharmaceutically acceptable salt, tautomer, or solvate thereof.

In some embodiments, the adenosine derivative is a compound having the structure:

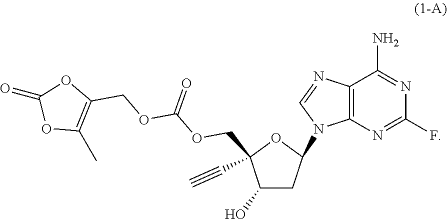 [SMILES: C#C[C@]1(COC(=O)OCc2oc(=O)oc2C)O[C@@H](n2cnc3c(N)nc(F)nc32)C[C@@H]1O]

The compound of formula (1-A) can also be referred to by a chemical name, e.g., ((2R,3S,5R)-5-(6-amino-2-fluoro-9H-purin-9-yl)-2-ethynyl-3-hydroxytetrahydro-furan-2-yl)methyl ((5-methyl-2-oxo-1,3-dioxol-4-yl)methyl) carbonate.

In some embodiments, the adenosine derivative is EFdA (4′-ethynyl-2-fluoro-2′-deoxyadenosine).

The HIV infection for treatment or prevention by the disclosed methods can be caused by wild-type HIV-1, NRTI-resistant HIV-1, HIV-2, HIV having M184V mutations, HIV having K65R, or multidrug resistant HIV. In some embodiments, the HIV infection is caused by HIV-1. In some embodiments, the HIV infection is an HIV-1 infection.

In some embodiments, to treat or prevent HIV infection, the subject is administered about 0.1 mg, about 0.2 mg, about 0.25 mg, about 0.3 mg, about 0.38 mg, about 0.4 mg, about 0.5 mg, about 0.6 mg, about 0.7 mg, about 0.75 mg, about 0.8 mg, about 0.9 mg, about 1.0 mg, about 1.1 mg, about 1.2 mg, about 1.25 mg, about 1.3 mg, about 1.4 mg, about 1.5 mg, about 1.6 mg, about 1.7 mg, about 1.75 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg, about 3.1 mg, about 3.2 mg, about 3.3 mg, about 3.4 mg, about 3.5 mg, about 3.6 mg, about 3.7 mg, about 3.8 mg, about 3.9 mg, 4.0 mg, about 4.1 mg, about 4.2 mg, about 4.3 mg, about 4.4 mg, about 4.5 mg, about 4.6 mg, about 4.7 mg, about 4.8 mg, about 4.9 mg, about 5.0 mg, about 6.0 mg, about 6.5 mg, about 7.0 mg, about 7.5 mg, about 8.0 mg. about 8.5 mg, about 9.0 mg, about 9.5 mg, or about 10 mg of the adenosine derivative. In some embodiments, the subject is administered 2.3 mg of the adenosine derivative. In some embodiments, the subject is administered 1.5 mg of the adenosine derivative. In some embodiments, the subject is administered 0.25 mg or 0.38 mg of the adenosine derivative. In some embodiments, the subject is administered 0.25 mg of the adenosine derivative. In some embodiments, the subject is administered 0.38 mg of the adenosine derivative. In some embodiments, the subject is administered a dose of the adenosine derivative that is equivalent to 0.25 mg of islatravir (EFdA). In some embodiments, the subject is administered a dose of the adenosine derivative that has the same molar amount as 0.25 mg of islatravir (EFdA).

In some embodiments, the subject is administered from about 0.1 mg to about 1.75 mg of the adenosine derivative, e.g., about 0.1 mg, about 0.2 mg, about 0.25 mg, about 0.3 mg, about 0.4 mg, about 0.5 mg, about 0.6 mg, about 0.7 mg, about 0.75 mg, about 0.8 mg, about 0.9 mg, about 1.0 mg, about 1.1 mg, about 1.2 mg, about 1.25 mg, about 1.3 mg, about 1.4 mg, about 1.5 mg, about 1.6 mg, about 1.7 mg, about 1.75 mg, inclusive of all ranges and values therebetween. In some embodiments, the subject is administered from about 1.3 mg to about 1.75 of the adenosine derivative. In some embodiments, the subject is administered from about 0.1 mg to about 0.75 mg of the adenosine derivative. In some embodiments, the subject is administered from about 0.25 mg to about 0.75 mg of the adenosine derivative. In some embodiments, the subject is administered from about 0.38 mg to about 0.75 mg of the adenosine derivative.

The adenosine derivative disclosed herein can be administered according to any dosing regimen effective for treating or preventing HIV infection in a subject. In some embodiments, the adenosine derivative is administered once every six months, once-month, or less frequently in an amount disclosed herein. In some embodiments, the adenosine derivative is administered once-monthly or less frequently in an amount disclosed herein. In some embodiments, the adenosine derivative is administered once-monthly, once-weekly, or once-daily in an amount disclosed herein. In some embodiments, the adenosine derivative is administered once-weekly or less frequently in an amount disclosed herein. In some embodiments, the adenosine derivative is administered once-weekly or once-daily in an amount disclosed herein.

In some embodiments, the adenosine derivative is administered once-weekly. In some embodiments, the once-weekly dose comprises from about 1 mg to about 25 mg of the adenosine derivative, inclusive of all ranges and values therebetween. In some embodiments, the once-weekly dose comprises from about 1 mg to about 10 mg of the adenosine derivative. In some embodiments, the once-weekly dose comprises from about 1 mg to about 2 mg of the adenosine derivative, e.g., about 1 mg, about 1.1 mg, about 1.2 mg, about 1.25, or about 2 mg. In some embodiments, the once-weekly dose comprises from about 1.25 mg to about 1.8 mg of the adenosine derivative. In some embodiments, the once-weekly dose comprises from about 1.3 mg to about 1.75 mg, e.g., about 1.3 mg, about 1.35 mg, about 1.4 mg, about 1.45 mg, about 1.5 mg, about 1.55 mg, about 1.6 mg, about 1.65 mg, about 1.7 mg, or about 1.75 mg of the adenosine derivative, inclusive of all ranges and values therebetween.

In some embodiments, the adenosine derivative is administered once-daily. In some embodiments, the once-daily dose comprises from 0.1 mg to about 5 mg of the adenosine derivative, inclusive of all ranges and values therebetween. In some embodiments, the once-daily dose comprises from about 0.1 mg to about 0.75 mg, e.g., about 0.1 mg, about 0.2 mg, about 0.3 mg, about 0.4 mg, about 0.5 mg, about 0.6 mg, about 0.7 mg, or about 0.75 mg of the adenosine derivative, inclusive of all ranges and values therebetween. In some embodiments, the once-daily dose comprises about 0.25 mg or 0.38 mg of the adenosine derivative. In some embodiments, the once-daily dose comprises about 0.25 mg of the adenosine derivative. the once-daily dose comprises about 0.38 mg of the adenosine derivative.

The adenosine derivative can be administered to the subject in need by any route of administration known in the art. In some embodiments, the adenosine derivative is administered to the subject orally, or by intramuscular (IM), subcutaneous (SC) or parenteral injection. In some embodiments, the adenosine derivative is administered orally. In some embodiments, oral administration is in the form of a tablet. In some embodiments, the tablet is administered once-weekly. In some embodiments, the tablet is administered once-daily.

In some embodiments of the methods provided herein, the adenosine derivative or pharmaceutically acceptable salt thereof is administered to the subject having HIV for a plurality of weeks. In some embodiments, the plurality of weeks can be for at least 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks or more weeks. In some embodiments, the plurality of weeks can be for at least 96 weeks.

The methods of the present disclosure can further comprise administering to the subject one or more anti-HIV agents. In some embodiments, the one or more anti-HIV agents is selected from atazanavir, atazanavir sulfate, bictegravir, cabotegravir, dolutegravir, doravirine, efavirenz, tenofovir disoproxil fumarate, tenofovir alafenamide, elvitegravir, etravirine, darunavir, a combination of darunavir and cobicistat, rilpivirine, lenacapavir, and combinations thereof. In some embodiments, the one or more anti-HIV agents is rilpivirine. In some embodiments, the one or more anti-HIV agents is doravirine. In some embodiments, the one or more anti-HIV agents is lenacapavir. Clinically approved dosages for the one or more anti-HIV agents can be suitable. The pharmaceutical composition and the one or more anti-HIV agents can be in separate formulations and can be administered to a subject simultaneously or sequentially. The adenosine derivative can also be formulated together with the one or more anti-HIV agents to form a single formulation that can be administered to a subject simultaneously. The pharmaceutical composition comprising the adenosine derivative and the one or more anti-HIV agents in separate formulations can also be mixed that can be administered to a subject simultaneously.

In some embodiments, the subject is treatment-naïve, i.e., an HIV-positive subject that has not been previously treated for HIV infection. In some embodiments, the subject has been previously treated for HIV infection. In some embodiments, the subject has been previously administered one or more other anti-HIV drugs. In some embodiments, the subject undergoing treatment for HIV infection is virologically suppressed from one or more other anti-HIV drugs.

In some embodiments, the subject undergoing treatment is at risk for total lymphocyte count reduction compared to a subject administered a control drug, such as tenofovir. In some embodiments, the subject undergoing treatment is at risk for total lymphocyte count reduction compared to a treatment-naïve subject. In some embodiments, the subject is at risk for CD4⁺ lymphocyte count reduction compared to a subject administered a control drug, such as tenofovir. In some embodiments, the subject is at risk for CD4⁺ lymphocyte count reduction compared to a treatment-naïve subject. In some embodiments, a subject administered an effective amount of an adenosine derivative disclosed herein for treating or preventing HIV infection exhibits a greater increase in total lymphocyte count compared to a subject administered a dose equivalent amount of islatravir (EFdA). In some embodiments, a subject administered an effective amount of an adenosine derivative disclosed herein for treating or preventing HIV infection exhibits a greater increase in CD4⁺ lymphocyte count compared to a subject administered a dose equivalent amount of islatravir (EFdA).

In some embodiments, the subject has a renal deficiency. In some embodiments, the subject has reduced expression of deoxycytidine kinase (DCK). In some embodiments, the subject has reduced expression of adenosine deaminase (AD). In some embodiments, the subject has reduced expression of purine nucleoside phosphorylase (PNP). In some embodiments, the subject has elevated expression of deoxycytidine kinase (DCK). In some embodiments, the subject has elevated expression of adenosine deaminase (AD). In some embodiments, the subject has elevated expression of purine nucleoside phosphorylase (PNP). In some embodiments, reduced or elevated expression level of DCK, AD, and PNP is determined by comparison to a treatment-naïve subject that is HIV-positive. In some embodiments, reduced or elevated expression level of DCK, AD, and PNP is determined by comparison to healthy subject (i.e., a subject that is HIV-negative). “Reduced expression”, as used herein, means lower RNA transcripts or lower activity as found in peripheral blook mononuclear cells (PBMCs) from patients. “Elevated expression”, as used herein, means elevated RNA transcripts or elevated activity as found in peripheral blook mononuclear cells (PBMCs) from patients. Assays for measuring reduced expression of AD, DCK, and PNP are known in the art, e.g., Turriziani et al. “Thymidine Kinase and Deoxycytidine Kinase Activity in Mononuclear Cells from Antiretroviral-naïve HIV Infected Patients” AIDS 2005, 19:473-479; Costa et al. “Biochemical Characterization of Adenosine Deaminase (CD26; EC 3.5.4.4) Activity in Human Lymphocyte-Rich Peripheral Blood Mononuclear Cell” Brazilian J. of Medical and Biological Research 2021, 54(8); and Murray et al. “Elevated Adenosine Deaminases and Purine Nucleoside Phosphorylase Activity in Peripheral Blood Null Lymphocytes From Patents with Acquired Immune Deficiency Syndrome” Blood 1985, Vol. 65, No. 6, 1318-1323, each of which is incorporated herein by reference in its entirety.

In some embodiments, the present disclosure provides a method of treating or preventing HIV infection, comprising administering to a subject in need thereof 0.25 mg of a compound having the structure:

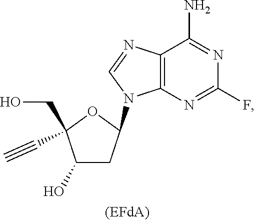 [SMILES: C#C[C@]1(CO)O[C@@H](n2cnc3c(N)nc(F)nc32)C[C@@H]1O]

or a pharmaceutically acceptable salt thereof, wherein the compound is administered orally to the subject once-daily in the form of a tablet.

In some embodiments, the EFdA (islatravir) is administered to the subject as a single agent. In some embodiments, the EFdA (islatravir) is administered to the subject in combination with one or more anti-HIV agents. In some embodiments, the one or more anti-HIV agents is selected from atazanavir, atazanavir sulfate, bictegravir, cabotegravir, dolutegravir, doravirine, efavirenz, tenofovir disoproxil fumarate, tenofovir alafenamide, elvitegravir, etravirine, darunavir, a combination of darunavir and cobicistat, rilpivirine, lenacapavir, and combinations thereof. In some embodiments, the one or more anti-HIV agents is doravirine. In some embodiments, the one or more anti-HIV agents is lenacapavir. Clinically approved dosages for the one or more anti-HIV agents can be suitable.

In some embodiments, the method of the present disclosure further comprises.
(a) measuring purine nucleoside phosphorylase (PNP) activity prior to administering the pharmaceutical composition to produce baseline PNP activity and after administering the pharmaceutical composition to produce post admin PNP activity from samples of the subject;
(b) producing PNP activity data by comparing the post admin PNP activity to the baseline PNP activity; and
(c) adjusting the effective dosage based on the PNP activity data so the post admin PNP activity is in a range of from 20% to 200% of the baseline PNP activity.

Percentage can be based on the baseline PNP activity. In some embodiments, the post admin PNP activity can be 20% to 200%, 30% to 200%, 40% to 200%, 50% to 200%, 60% to 200%, 70% to 200%, 80% to 200%, 90% to 200%, 100% to 200%, 50% to 150%, 50% to 120%, 50% to 100%, 50% to 90%, 50% to 80%, 50% to 70%, 50% to 60% of the baseline PNP activity. In some embodiments, the effective dosage can be reduced if the post admin PNP activity is less than 100%, less than 90%, less than 80%, less than 70%, less than 60%, less than 50%, less than 40%, less than 30%, or less than 20%, of the baseline PNP activity. In some embodiments, the effective dosage may be increased if the subject exhibits variations of the PNP activities or responses to the treatment with the pharmaceutical composition, such as having the post admin PNP activity is more than 100%, more than 110%, more than 120%, more than 130%, more than 140%, more than 150%, or more than 200% of the baseline PNP activity.

Not wishing to be bound by a particular theory or mechanism, Applicants unexpectedly discovered that the purine nucleoside phosphorylase (PNP) activity can be an indication for adjusting dosages of the adenosine derivative disclosed herein. Adjusting the effective dosage of the adenosine derivative based on the inhibition of the PNP activity can help to minimize exposure related side effects such as decrease in total lymphocyte and CD4+ T-cell counts and to improve overall treatment outcome and quality of life to the subject.

In some embodiments, the method further comprises measuring total lymphocyte counts, CD4+ T-cell counts, or combination thereof prior to administering the pharmaceutical composition to produce baseline diagnostic data and after administering the pharmaceutical composition to produce post admin diagnostic data from samples of the subject.

In some embodiments, the method comprises measuring purine nucleoside phosphorylase (PNP) activity, total lymphocyte counts, CD4+ T-cell counts, or combination thereof prior to administering the pharmaceutical composition to produce baseline diagnostic data and after administering the pharmaceutical composition to produce post admin diagnostic data from samples of the subject
producing diagnostic data by comparing the post admin diagnostic data to the baseline diagnostic data; and
adjusting the effective dosage based on the diagnostic data so the post admin diagnostic data is in a range of from 20% to 200% of the baseline diagnostic data.

In some embodiments, the method can comprise measuring purine nucleoside phosphorylase (PNP) activity and total lymphocyte counts from a sample of the subject. In some embodiments, the process can comprise measuring purine nucleoside phosphorylase (PNP) activity and total CD4+ T-cell counts from a sample of the subject. In some embodiments, the process can comprise measuring purine nucleoside phosphorylase (PNP) activity, total lymphocyte counts and total CD4+ T-cell counts from a sample of the subject.

In some embodiments, the present disclosure is further directed to a process for diagnosing and subsequently treating or preventing HIV infection in a subject in need thereof, the process comprising measuring purine nucleoside phosphorylase (PNP) activity from a sample of the subject to produce individual baseline PNP activity of the subject.

In some embodiments, the process further comprises measuring total lymphocyte counts, CD4+ T-cell counts, or combination thereof from samples of the subject. In some embodiments, the process comprises measuring purine nucleoside phosphorylase (PNP) activity and total lymphocyte counts from a sample of the subject to produce individual baseline diagnostic data of the subject. In some embodiments, the process comprises measuring purine nucleoside phosphorylase (PNP) activity and total CD4+ T-cell counts from a sample of the subject to produce individual baseline diagnostic data of the subject. In some embodiments, the process comprises measuring purine nucleoside phosphorylase (PNP) activity, total lymphocyte counts and total CD4+ T-cell counts from a sample of the subject to produce individual baseline diagnostic data of the subject. In some embodiments, the process comprises measuring purine nucleoside phosphorylase (PNP) activity, and optionally, measuring total lymphocyte counts and total CD4+ T-cell counts from a sample of the subject to produce individual baseline diagnostic data of the subject.

The process can further comprise:
comparing the individual baseline PNP activity to pre-stored normalized baseline PNP activity; and
administering an effective dosage of an adenosine derivative to the subject if the individual baseline PNP activity is at least 10% or more of the pre-stored normalized baseline PNP activity.

In some embodiments, the process comprises administering an effective dosage of an adenosine derivative to the subject if the individual baseline PNP activity is at least 20% or more of the pre-stored normalized baseline PNP activity.

In some embodiments, the process comprises administering an effective dosage of an adenosine derivative to the subject if the individual baseline diagnostic data is at least 10% or 20% or more of the pre-stored normalized baseline diagnostic data. The purine nucleoside phosphorylase (PNP) activity, total lymphocyte counts and total CD4+ T-cell counts is compared individually. In some embodiments, the process further comprises comparing said individual baseline diagnostic data to pre-stored normalized baseline diagnostic data; and administering an effective dosage of an adenosine derivative to said subject if said individual baseline diagnostic data is at least 20% or more of the pre-stored normalized baseline diagnostic data.

When the process comprises administering a pharmaceutical composition, the pharmaceutical composition can comprise at least a nucleotide, a nucleotide derivative, an adenosine derivative, at least an integrase inhibitor, at least a nucleoside RT inhibitors (NRTI), at least a non-nucleoside reverse transcriptase inhibitor (NNRTI), at least a protease inhibitor, or a combination thereof.

In some embodiments, the nucleoside RT inhibitors (NRTI) can include 3′-azido-3′-deoxythymidine (AZT, also known as Zidovudine (ZDV), azidothymidine (AZT)), 2′,3′-dideoxyinosine (ddl), 2′,3′-dideoxycytidine (ddC), d4T, 3TC, abacavir, emtricitabine, and tenofovir disoproxil fumarate. The non-nucleoside RT inhibitors (NNRTI) can include nevirapine, delavirdine, efavirenz, rilpivirine and doravirine.

In some embodiments, the pharmaceutical composition comprises an adenosine derivative.

In some embodiments, the adenosine derivative is a compound of formula (1):

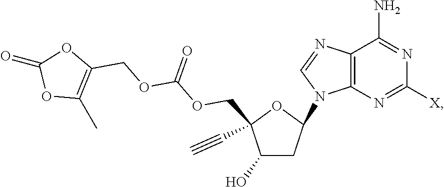

wherein X is a halogen atom, 4′-ethynyl-2-fluoro-2′-deoxyadenosine (EFdA), a pharmaceutically acceptable salt, stereoisomer, tautomer, solvate thereof, or a combination thereof.

In some embodiments, the adenosine derivative comprises formula (1-A):

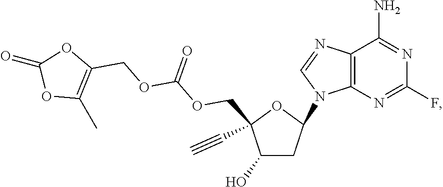 [SMILES: C#C[C@]1(COC(=O)OCc2oc(=O)oc2C)O[C@@H](n2cnc3c(N)nc(F)nc32)C[C@@H]1O]

a pharmaceutically acceptable salt, stereoisomer, tautomer, or solvate thereof, or a combination thereof.

In some embodiments, the process is suitable for treatment of a subject with any pharmaceutical compositions that comprises one or more nucleotide derivatives or analogues having or suspected having potent PNP inhibition activities. In some embodiments, purine nucleoside, purine nucleoside analogues, purine nucleoside analogues modified at various positions of both the heterocyclic base and carbohydrate residues including 8-mercapto-acyclovir, 8-bromo-9-(3,4-hydroxy-butyl)guanine is suitable.

In some embodiments, this process for diagnosis and treatment or prevention of HIV infection is suitable for treatment of a subject with any pharmaceutical compositions that comprises one or more adenosine derivatives. In some embodiments, the process is suitable for treatment of a subject with the pharmaceutical composition disclosed herein. In some embodiments, the process is suitable for treatment of a subject with EFdA (4′-ethynyl-2-fluoro-2′-deoxyadenosine, also known as MK-8591 or islatravir.

In some embodiments, the adenosine derivative is selected from the group consisting of formula (1-A):

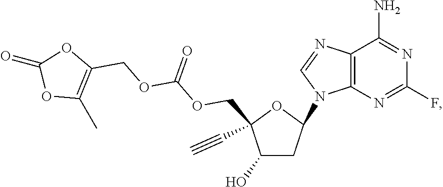 [SMILES: C#C[C@]1(COC(=O)OCc2oc(=O)oc2C)O[C@@H](n2cnc3c(N)nc(F)nc32)C[C@@H]1O]

EFdA (4′-ethynyl-2-fluoro-2′-deoxyadenosine, also known as MK-8591 or islatravir), and a combination thereof.

In some embodiments, the individual baseline PNP activity is in a range of from 20% to 200% of the pre-stored normalized baseline PNP activity.

The PNP activity is measured as enzymatic activity, PNP gene expression level, such as mRNA level, the PNP gene genomic DNA, or a combination thereof. Typical enzymatic assay for the purine nucleoside phosphorylase that is known to hose skilled in the art is suitable. Messenger RNA (mRNA) level of the purine nucleoside phosphorylase is measured with reverse transcription PCT (RT-PCR) or quantitative RT-PCT (qRT-PCR) using suitable primers. The PNP gene genomic DNA level is measured with typical PCR or quantitative PCR (qPCR) using suitable primers. Certain mutations or single nucleotide polymorphisms (SNPs) may impact PNP activity. The PNP gene genomic DNA level and the presence of certain mutations is an indication on the suitability of the subject for the treatment with the pharmaceutical composition of this disclosure.

In some embodiments, each of the individual baseline PNP activity, the pre-stored normalized baseline PNP activity, or a combination thereof, is measured based on PNP enzymatic activity, PNP mRNA, PNP genomic DNA, PNP gene mutations, PNP gene single nucleotide polymorphisms (SNPs), or a combination thereof.

When the individual baseline PNP activity is less than 20% of the pre-stored normalized baseline PNP activity, the subject will not be administered with the pharmaceutical composition. The PNP activity of the subject is a diagnostic indication for determining the suitability of the subject for the pharmaceutical composition to reduce side effect and to improve treatment to the subject.

The normalized baseline PNP activity is measured from multiple normal healthy individuals and normalized with traditional mathematically methods, such as average, root average, mean, or any other suitable methods as long as the individual baseline PNP activity is compared to normalized baseline PNP activity produced with the same method. The normalized baseline PNP activity is pre-stored such as in a digital database, a printed material, a display, or a combination thereof.

The pre-stored normalized baseline diagnostic data comprises normalized baseline PNP activity, total lymphocyte counts and total CD4+ T-cell counts from samples of multiple subjects and normalized as describe herein or by any method known to those skilled in the art.

In some embodiments, commercial fluorometric purine nucleoside phosphorylase activity assay kit is used. In some embodiments, an assay can measure fluorescent product from hypoxanthine formed from the breakdown of inosine via a multi-step reaction, resulting in the generation of an intermediate that reacts with a PNP Probe. In some embodiments, the fluorescent product is measured at Ex/Em=535/587 nm to determine the PNP activity.

This disclosure is further directed to a use of an adenosine derivative and one or more pharmaceutically acceptable carriers for manufacturing a medicament for treating a disease, wherein the adenosine derivative can have a formula (1):

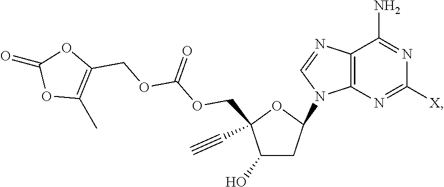

a pharmaceutically acceptable salt, stereoisomer, tautomer, or solvate thereof; and X is a halogen atom.

In some embodiments, X is F and the adenosine derivative is a compound of formula (1-A):

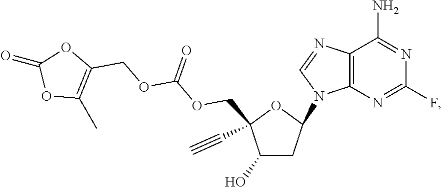 [SMILES: C#C[C@]1(COC(=O)OCc2oc(=O)oc2C)O[C@@H](n2cnc3c(N)nc(F)nc32)C[C@@H]1O]

a pharmaceutically acceptable salt, stereoisomer, tautomer, or solvate thereof.

The medicament is formulated as an implant, a controlled release implant, an oral suspension, an oral tablet, or an injectable composition suitable for intramuscular (IM), subcutaneous (SC) or parenteral injections.

In some embodiments, the medicament is formulated to comprise in a range of from 0.1 mg to 10 mg of the adenosine derivative in a single dosage. In some embodiments, the medicament is formulated to comprise in a range of from 0.1 mg to 7.5 mg of the adenosine derivative in a single dosage. In some embodiments, the medicament is formulated to comprise in a range of from 0.1 mg to 5.0 mg of the adenosine derivative in a single dosage. In some embodiments, the medicament is formulated to comprise in a range of from 0.1 mg to 2.5 mg of the adenosine derivative in a single dosage. In some embodiments, the medicament is formulated to comprise in a range of from 0.1 mg to 1.0 mg of the adenosine derivative in a single dosage.

The disease is an HIV infection. In some embodiments, the HIV infection is caused by wild-type HIV-1, NRTI-resistant HIV-1, HIV-2, HIV having M184V mutations, HIV having K65R, or multidrug resistant HIV.

The present disclosure is further directed to a method for the prevention of infection in a subject in need thereof, the method comprising administering the subject an effective dosage of any one of the pharmaceutical compositions or the therapeutical compositions disclosed herein, wherein the subject is free from detectable symptoms of the infection. In some embodiments, the infection comprises a disease selected from Acquired Immune Deficiency Syndrome (AIDS), an infection of wild-type HIV-1, NRTI-resistant HIV-1, HIV-2, HIV having M184V mutations, HIV having K65R, multidrug resistant HIV, an RNA virus infection, or a combination thereof.

The detectable symptoms can include, but are not limited to, symptoms of Acquired Immune Deficiency Syndrome (AIDS), symptoms of infection of HIV viruses comprising wild-type HIV-1, NRTI-resistant HIV-1, HIV-2, HIV having M184V mutations, HIV having K65R, multidrug resistant HIV, or a combination thereof. The detection of the HIV viruses is done by PCR, reverse PCR, immunodetection of an antigen or an antibody related to AIDS or HIV.

Without being bound by any particular theory, an advantage of the adenosine derivatives disclosed herein can have a fast conversion to the target drug. Greater than about 60% of the adenosine derivatives of the present disclosure is converted to the target drug within about 30 min in contact with human plasma or human liver enzymes.

Further provided herein are compositions comprising an adenosine derivative or a pharmaceutically acceptable salt thereof for use in a method for treating an HIV infection in a subject according to any one of the methods described herein.

### Pharmaceutical Compositions

The present disclosure provides pharmaceutical compositions comprising an adenosine derivative; and one or more pharmaceutically acceptable carriers; wherein the pharmaceutical composition comprises from 0.1 mg to 25 mg of the adenosine derivative.

In some embodiments, the adenosine derivative is a compound of formula (1):

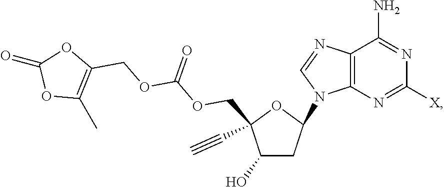

a pharmaceutically acceptable salt, stereoisomer, tautomer, or solvate thereof; wherein X is a halogen atom. In some embodiments of formula (1), the X is a halogen atom selected from the group consisting of fluorine, chlorine, bromine and iodine. In some embodiments, X is F. In some embodiments, X is Cl. In some embodiments, X is Br.

In some embodiments, X is F and the adenosine derivative is a compound of formula (1-A):

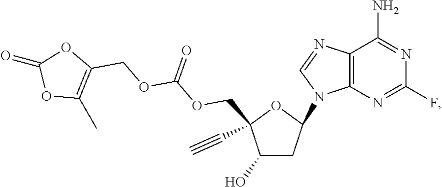 [SMILES: C#C[C@]1(COC(=O)OCc2oc(=O)oc2C)O[C@@H](n2cnc3c(N)nc(F)nc32)C[C@@H]1O]

or a pharmaceutically acceptable salt, stereoisomer, tautomer, or solvate thereof.

In some embodiments, the pharmaceutical composition is formulated as an implant, a controlled release implant, an oral suspension, an oral tablet, or an injectable composition suitable for intramuscular (IM), subcutaneous (SC) or parenteral injections. In some embodiments, the pharmaceutical composition is formulated as an oral tablet.

In some embodiments, the pharmaceutical composition is administered to a subject once-daily (QD) or from once per week (Q1W) to once every 8 weeks (Q8W). In some embodiments, the pharmaceutical composition is administered to a subject once per week (Q1W), once every two weeks (Q2W), once every 3 weeks (Q3W), once every 4 weeks (Q4W), once every 5 weeks (Q5W), once every 6 weeks (Q6W), once every 7 weeks (Q7W), or once every 8 weeks (Q8W). In some embodiments, the pharmaceutical composition is administered to a subject once-daily (QD) or once-weekly.

In some embodiments, the pharmaceutical composition is administered to a subject in a range of from once per month (Q1M) to once every 12 months (Q12M). In some embodiments, the pharmaceutical composition is administered to a subject once per month (Q1M), once every two months (Q2M), once every 3 months (Q3M), once every 4 months (Q4M), once every 5 months (Q5M), once every 6 months (Q6M), once every 7 months (Q7M), once every 8 months (Q8M), once every 9 months (Q9M), once every 10 months (Q10M), once every 11 months (Q11M), and once every 12 months (Q12M).

In some embodiments, the pharmaceutical composition comprises from 0.1 mg to 10 mg, e.g., 0.1 mg, 0.2 mg, 0.25 mg, 0.3 mg, 0.38 mg, 0.4 mg, 0.5 mg, 0.6 mg, 0.7 mg, 0.75 mg, 0.8 mg, 0.9 mg, 1.0 mg, 1.1 mg, 1.2 mg, 1.3 mg, 1.4 mg, 1.5 mg, 1.6 mg, 1.7 mg, 1.75 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 4.1 mg, 4.2 mg, 4.3 mg, 4.4 mg, 4.5 mg, 4.6 mg, 4.7 mg, 4.8 mg, 4.9 mg, 5.0 mg, 6.0 mg, 6.5 mg, 7.0 mg, 7.5 mg, 8.0 mg. 8.5 mg, 9.0 mg, 9.5 mg, or 10 mg, of the adenosine derivative in a single dosage, inclusive of all ranges and values therebetween. The term “single dosage” refers to a dosage format suitable for administering to a person. In some embodiments, a single dosage is in the form of a tablet, such as one or more tablets that is taken by a person at once. In some embodiments, a single dosage is injectable solution or suspension that is injected to a person in a single injection or one or more injections at once. In some embodiments, a single dosage is an implant that is delivered to a person at once. In some embodiments, the pharmaceutical composition comprises from 0.1 mg to 1.75 mg of the adenosine derivative in a single dosage. In some embodiments, the pharmaceutical composition comprises 0.25 mg of the adenosine derivative in a single dosage. In some embodiments, the pharmaceutical composition comprises 0.38 mg of the adenosine derivative in a single dosage. In some embodiments, the pharmaceutical composition comprises a dose equivalent of 0.25 mg of islatravir. In some embodiments, the subject is administered a dose of the adenosine derivative that has the same molar amount as 0.25 mg of islatravir.

In some embodiments, the pharmaceutical composition comprises from 0.1 mg to 10 mg, 0.1 mg to 9.5 mg, 0.1 mg to 9.0 mg, 0.1 mg to 8.5 mg, 0.1 mg to 8.0 mg, 0.1 mg to 7.5 mg, 0.1 mg to 7.0 mg, 0.1 mg to 6.5 mg, 0.1 mg to 6.0 mg, 0.1 mg to 5.5 mg, 0.1 mg to 5.0 mg, 0.1 mg to 4.5 mg, 0.1 mg to 4.0 mg, 0.1 mg to 3.0 mg, 0.1 mg to 2.0 mg, 0.1 mg to 1.5 mg, 0.1 mg to 1.0 mg, 0.1 mg to 0.75 mg, 0.1 mg to 0.5 mg, 0.1 mg to 0.4 mg, 0.1 mg to 0.3 mg, or 0.1 mg to 0.2 mg, of the adenosine derivative in a single dosage for administering to a subject once per week (Q1W).

In some embodiments, the pharmaceutical composition comprises from 0.1 mg to 0.75 mg, 0.1 mg to 0.7 mg, 0.1 mg to 0.65 mg, 0.1 mg to 0.6 mg, 0.1 mg to 0.55 mg, 0.1 mg to 0.50 mg, 0.1 mg to 0.45 mg, 0.1 mg to 0.4 mg, 0.1 mg to 0.3 mg, or 0.1 mg to 0.25 mg of the adenosine derivative in a single dosage for administering to a subject once per day (QD).

The pharmaceutical compositions disclosed herein are useful for treating HIV infection. In some cases, the HIV infection is caused by wild-type HIV-1, NRTI-resistant HIV-1, HIV-2, HIV having M184V mutations, HIV having K65R, or multidrug resistant HIV. In some embodiments, the HIV infection is an HIV-1 infection.

The adenosine derivative of the present disclosure is a prodrug that can have no or limited activity in its original form shown herein and is metabolized in vivo to exhibit the desired activity of a target drug including a reverse transcriptase inhibitor activity, a reverse transcriptase chain terminator activity, DNA translocation inhibitor activity, or a combination thereof.

Not wishing to be bound by a particular mechanism or theory, Applicant discovered that adenosine derivatives of the present disclosure can be metabolized in vivo to produce a target drug comprising a compound or a mixture of compounds that can have reverse transcriptase inhibitor and other antiviral activities.

In some embodiments, the target drug is a compound of formula (T-1):

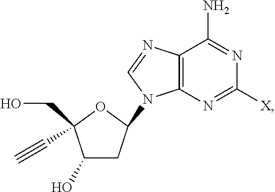

an isomer thereof, or a pharmaceutically acceptable salt thereof. In some embodiments, X is a halogen selected from the group consisting of F, Cl, Br, and I. In some embodiments, X is F or Cl. In some embodiments, X is F. In some embodiments, X is Cl.

In some embodiments, X is F and the target drug is a compound of

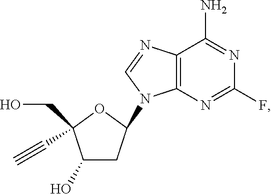 [SMILES: C#C[C@]1(CO)O[C@@H](n2cnc3c(N)nc(F)nc32)C[C@@H]1O]

an isomer thereof, or a pharmaceutically acceptable salt thereof. The target drug is also known as (2R,3S,5R)-5-(6-amino-2-fluoro-9H-purin-9-yl)-2-ethynyl-2-(hydroxymethyl)tetrahydrofuran-3-ol (also referred to as 4′-ethynyl-2-fluoro-2′-deoxyadenosine, herein “EFdA” or “Islatravir”), or a pharmaceutically acceptable salt thereof. The terms 4′-ethynyl-2-fluoro-2′-deoxyadenosine, “EFdA” or “Islatravir” may be used herein interchangeably.

In some embodiments, the target drug is a degradation or metabolized product of the compound (T-1) or (T-1A) (EFdA). In some embodiments, the target drug is EFdA diphosphate (EFdA-DP)

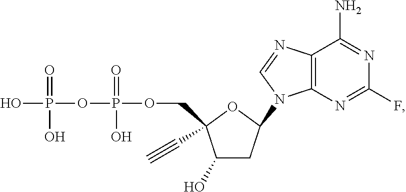 [SMILES: C#C[C@]1(COP(=O)(O)OP(=O)(O)O)O[C@@H](n2cnc3c(N)nc(F)nc32)C[C@@H]1O]

EFdA triphosphate (EFdA-TP)

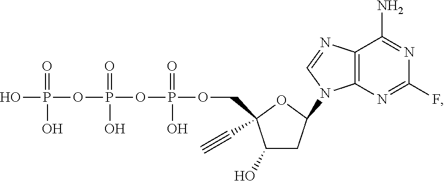 [SMILES: C#C[C@]1(COP(=O)(O)OP(=O)(O)OP(=O)(O)O)O[C@@H](n2cnc3c(N)nc(F)nc32)C[C@@H]1O]

or a combination thereof.

The target drug is measured from a specimen of the subject. The specimen is a blood sample, human peripheral blood mononuclear cells (hPBMCs), a urine sample, a body fluid sample, a tissue sample or a combination thereof from the subject, such as a patient.

In some embodiments, EFdA triphosphate (EFdA-TP) is measured from human peripheral blood mononuclear cells (hPBMCs). In some embodiments, a steady state C_{trough} of 4′-ethynyl-2-fluoro-2′-deoxyadenosine triphosphate (EFdA-TP) is measured from human peripheral blood mononuclear cells (hPBMCs). In some embodiments, an in vivo steady state C_{trough} of 4′-ethynyl-2-fluoro-2′-deoxyadenosine triphosphate (EFdA-TP) is in a range of from 0.01 to 5 in one example, 0.01 to 4 in another example, 0.01 to 3 in another example, 0.01 to 2 in another example, 0.01 to 1 in another example, 0.01 to 0.5 in another example, 0.05 to 4 in another example, 0.1 to 2 in yet another example, 0.1 to 1.5 in yet another example, 0.1 to 0.5 in yet another example, pmol/10⁶ human peripheral blood mononuclear cells (hPBMCs).

In some embodiments, the pharmaceutical composition is formulated to produce an in vivo steady state C_{trough} of 4′-ethynyl-2-fluoro-2′-deoxyadenosine triphosphate (EFdA-TP) in a range of from 0.01 to 5.0 pmol/10⁶ human peripheral blood mononuclear cells (hPBMCs).

In some embodiments, the pharmaceutical composition is formulated to produce an in vivo steady state C_{trough} of 4′-ethynyl-2-fluoro-2′-deoxyadenosine triphosphate (EFdA-TP) of 0.01 to 2 pmol/10⁶ human peripheral blood mononuclear cells (hPBMCs) for switching maintenance therapy, i.e., a person who has been subject to treatment of one or more HIV infection treatment drugs prior be administered with the pharmaceutical composition disclosed herein.

In some embodiments, the pharmaceutical composition is formulated to produce an in vivo steady state C_{trough} of 4′-ethynyl-2-fluoro-2′-deoxyadenosine triphosphate (EFdA-TP) of 0.01 to 0.5 pmol/10⁶ human peripheral blood mononuclear cells (hPBMCs) for treatment-naïve therapy, i.e., a person who has not been exposed to treatment of one or more HIV infection treatment drugs prior be administered with the pharmaceutical composition disclosed herein.

An adenosine derivative of the present disclosure can comprise one or more isomers thereof. An isomer can comprise a chiral isomer, also known as stereoisomer, that comprises one or more chiral centers, a tautomer that can interconvert via the relocation of a proton or other atom, such as amino isomer, imino isomer, or a combination thereof. In examples, an adenosine derivative can have an amino isomer, an imino isomer or a combination thereof. In further examples, an adenosine derivative can comprise enantiomers, diastereomers and cis/trans isomers, tautomers or a combination thereof. An isomer that can have reverse transcriptase inhibitor (RTI) activity in vivo is particularly preferred.

In some embodiments, the pharmaceutical composition further comprises one or more anti-HIV agents selected from atazanavir, atazanavir sulfate, bictegravir, cabotegravir, dolutegravir, doravirine, efavirenz, tenofovir disoproxil fumarate, tenofovir alafenamide, elvitegravir, etravirine, darunavir, a combination of darunavir and cobicistat, rilpivirine, one or more capsid (CA) inhibitors, lenacapavir, and a combination thereof. In some embodiments, the one or more anti-HIV agent is rilpivirine. In some embodiments, the one or more anti-HIV agent is doravirine. In some embodiments, the one or more anti-HIV agent is lenacapavir. Clinically approved dosages for the one or more anti-HIV agents is suitable.

In some embodiments, the pharmaceutical composition of the present disclosure comprises an adenosine derivative and the one or more anti-HIV agents in a single formulation that is administered to a subject together. The pharmaceutical composition of the present disclosure can comprise the adenosine derivative and the one or more anti-HIV agents in separate formulations that is administered to a subject simultaneously or sequentially. The pharmaceutical composition of the present disclosure can also be mixed together with one or more anti-HIV agents in separate formulations that is administered to a subject simultaneously.

The pharmaceutical composition of the present disclosure is formulated as an implant, a controlled release implant, an oral suspension, an oral tablet, or an injectable composition suitable for intramuscular (IM), subcutaneous (SC) or parenteral injections. In some embodiments, pharmaceutical composition is formulated into an injectable solution. In some embodiments, pharmaceutical composition is formulated into an injectable solution that can form gel when in contact with an aqueous solution. In some embodiments, pharmaceutical composition is formulated into an injectable solution that can form gel implant in situ in the body of a subject in need thereof. In some embodiments, pharmaceutical composition is formulated as an implant or a controlled release implant that cam form in situ implant upon injection into a subject in need thereof.

The pharmaceutical composition of the present disclosure can comprise one or more pharmaceutically acceptable carriers.

Non-limiting examples of pharmaceutically acceptable carriers can include pharmaceutical excipients such as surfactant, emulsifier, filler, carrier, isotonicifier, dispersing agent, viscosity modifier, resuspending agent, buffer or a combination thereof. Pharmaceutical excipients typically do not have properties of a medicinal or drug active ingredient, such as those known as active pharmaceutical ingredient (API) and are typically used to streamline the manufacture process or packaging of the active ingredients, or to deliver an API to a patient or other subject. Pharmaceutical acceptable carrier, excipients or inactive ingredients from the Inactive Ingredients Database available from US FDA (https://www.fda.gov/drugs/drug-approvals-and-databases/inactive-ingredients-database-download) can be suitable. Some of Generally Recognized As Safe (GRAS) food substances available form US FDA's GRAS Substances (SCOGS) Database (https://www.fda.gov/food/generally-recognized-safe-gras/gras-substances-scogs-database) can also be suitable.

In some embodiments of the present disclosure, the pharmaceutically acceptable carrier comprises acacia, animal oils, benzyl alcohol, benzyl benzoate, calcium stearate, carbomers, cetostearyl alcohol, cetyl alcohol, cholesterol, cyclodextrins, dextrose, diethanolamine, emulsifying wax, ethylene glycol palmitostearate, glycerin, glycerin monostearate, glycerol stearate, glyceryl monooleate, glyceryl monostearate, hydrous, histidine, hydrochloric acid, hydroxypropyl cellulose, hydroxypropyl-β-cyclodextrin (HPBCD), hypromellose (hydroxypropyl methylcellulose (HPMC)), lanolin, lanolin alcohols, lecithin, medium-chain triglycerides, metallic soaps, methylcellulose, mineral oil, monobasic sodium phosphate, monoethanolamine, oleic acid, polyyethylene glycols (PEG 3350, PEG 4000, PEG 6000), polyoxyethylene-polyoxypropylene copolymer (poloxamer), polyoxyethylene alkyl ethers, polyoxyethylene castor oil, polyoxyethylene castor oil derivatives, polyoxyethylene sorbitan fatty acid esters, polyoxyethylene stearates, polysorbate, polyoxyethylene (20) sorbitan monolaurate (Tween 20, Polysorbate 20), polyoxyethylene (20) sorbitan monooleate (Tween 80, Polysorbate 80), povidone, propylene glycol alginate, saline, sodium chloride, sodium citrate, sodium citrate dihydrate, sodium hydroxide, sodium lauryl sulfate, sodium phosphate monobasic, sodium phosphate dibasic, sorbitan esters, stearic acid, stearyl alcohol, sunflower oil, tragacanth, triethanolamine, vegetable oils, water, xanthan gum, or combinations thereof.

In further embodiments, the pharmaceutical acceptable carrier comprises dextrose, glycerin, histidine, hydrochloric acid, hydroxypropyl cellulose, hydroxypropyl-β-cyclodextrin (HPBCD), hypromellose (hydroxypropyl methylcellulose (HPMC)), polyoxyethylene (20) sorbitan monolaurate (Tween 20, Polysorbate 20), polyyethylene glycols (PEG 400, PEG 3350, PEG 4000, PEG 6000), polyoxyethylene-polyoxypropylene copolymer (Poloxamer 188, Poloxamer 407), polyoxyethylene (20) sorbitan monooleate (Tween 80, Polysorbate 80), saline, sodium chloride, sodium citrate, sodium citrate dihydrate, sodium lauryl sulfate, sodium phosphate monobasic, sodium phosphate dibasic, or a combination thereof.

In some embodiments, the pharmaceutical composition of the present disclosure comprises an effective dosage of the adenosine derivative to produce in a range of from 0% to 90% inhibition of purine nucleoside phosphorylase (PNP) as assayed in vivo or in vitro. The PNP activity can be measured as enzymatic activity, PNP gene expression level, such as mRNA level, the PNP gene genomic DNA, or a combination thereof, as described later in this disclosure.

In some embodiments, purine nucleoside phosphorylase (PNP) activity can be measured prior to (baseline PNP activity) and after administering the pharmaceutical composition (post admin PNP activity) from samples of the subject. The post admin PNP activity can be compared to the baseline PNP activity. Percentage of inhibition of purine nucleoside phosphorylase (PNP) activity can be determined by comparing the post admin PNP activity to the baseline PNP activity, or by comparing individual post admin PNP activity to pre-stored normalized baseline PNP activity, as described hereafter in this disclosure.

In some embodiments, the pharmaceutical composition of this disclosure can be formulated to comprise an effective dosage of the adenosine derivative in the single dosage to produce in a range of from 0% to 90%, 0% to 80%, 0% to 70%, 0% to 60%, 0% to 50%, 0% to 40%, 0% to 30%, 0% to 20%, 0% to 10%, or 0% to 5% inhibition of purine nucleoside phosphorylase (PNP) activity. In some embodiments, the pharmaceutical composition of this disclosure can be formulated to comprise an effective dosage of the adenosine derivative in the single dosage to produce less than 5%, 10%, 20%, or 30% inhibition of purine nucleoside phosphorylase (PNP) activity.

In some embodiments, the pharmaceutical composition is formulated based on the process described hereafter for adjusting the effective dosage based on the PNP activity data so the post-admin PNP activity is in a range of from 20% to 200% of the baseline PNP activity. The percentage of inhibition and percentage of activity can be converted by traditional means, for example, when a post admin PNP activity is about 90% of the baseline PNP activity, the adenosine derivative in the single dosage can be considered to produce about 10% inhibition.

### NUMBERED EMBODIMENTS

1. A pharmaceutical composition comprising:
   an adenosine derivative or a pharmaceutically acceptable salt thereof; and
   one or more pharmaceutically acceptable carriers;
wherein said pharmaceutical composition comprises from 0.01 mg to 25 mg of said adenosine derivative.
2. The pharmaceutical composition of embodiment 1, wherein said adenosine derivative has the structure of formula (1):

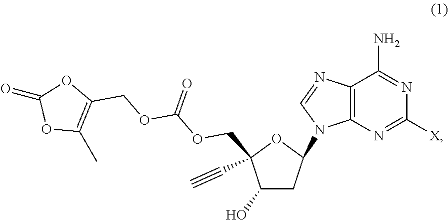

a pharmaceutically acceptable salt thereof, wherein X is a halogen atom.
3. The pharmaceutical composition of embodiment 2, wherein X is F and said adenosine derivative has the structure of formula (1-A):

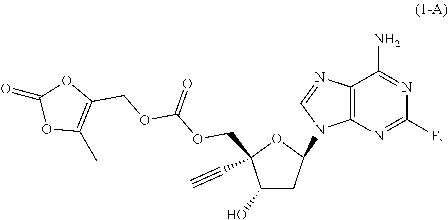 [SMILES: C#C[C@]1(COC(=O)OCc2oc(=O)oc2C)O[C@@H](n2cnc3c(N)nc(F)nc32)C[C@@H]1O]

a pharmaceutically acceptable salt thereof.
4. The pharmaceutical composition of any one of embodiments 1-3, wherein said pharmaceutical composition is formulated as an implant, a controlled release implant, an oral suspension, an oral tablet, or an injectable composition suitable for intramuscular (IM), subcutaneous (SC) or parenteral injections.
5. The pharmaceutical composition of any one of embodiments 1-4, wherein said pharmaceutical composition is formulated to produce an in vivo steady state C_{trough} of 4′-ethynyl-2-fluoro-2′-deoxyadenosine triphosphate (EFdA-TP) of 0.01 to 2 pmol/10⁶ human peripheral blood mononuclear cells (hPBMCs) for switching maintenance therapy.
6. The pharmaceutical composition of any one of embodiments 1-4, wherein said pharmaceutical composition is formulated to produce an in vivo steady state C_{trough} of 4′-ethynyl-2-fluoro-2′-deoxyadenosine triphosphate (EFdA-TP) of 0.01 to 0.5 pmol/10⁶ human peripheral blood mononuclear cells (hPBMCs) for treatment-naïve therapy.
7. The pharmaceutical composition of any one of embodiments 1-6 further comprising one or more anti-HIV agents selected from atazanavir, atazanavir sulfate, bictegravir, cabotegravir, dolutegravir, doravirine, efavirenz, tenofovir disoproxil fumarate, tenofovir alafenamide, elvitegravir, etravirine, darunavir, a combination of darunavir and cobicistat, rilpivirine, one or more capsid (CA) inhibitors, lenacapavir, and a combination thereof.
8. The pharmaceutical composition of any one of embodiments 1-7, wherein said pharmaceutical composition is comprises an effective dosage of said adenosine derivative in a single dosage to produce in a range of from 0% to 90% inhibition of purine nucleoside phosphorylase (PNP) as assayed in vivo or in vitro.
9. A method for treatment or prevention of HIV infection in a subject in need thereof, said method comprising administering an effective dosage of said pharmaceutical composition of any one of embodiments 1-8.
10. The method of embodiment 9, wherein said pharmaceutical composition is formulated as an implant, a controlled release implant, an oral suspension, an oral tablet, or an injectable composition suitable for intramuscular (IM), subcutaneous (SC) or parenteral injections.
11. The method of embodiment 10, wherein said pharmaceutical composition is administered to said subject once-daily (QD) or once-weekly (Q1W) to once every 8 weeks (Q8W).
11a. The method of embodiment 10 or 11, wherein said pharmaceutical composition is administered to said subject once-daily (QD) or once-weekly (Q1W).
12. The method of embodiment 10, wherein said pharmaceutical composition is administered to said subject in a range of from once per month to once every 12 months.
13. The method of any one of embodiments 9-12, wherein said effective dosage is in a range of from 0.1 mg to 10 mg of said adenosine derivative once per week (Q1W).
14. The method of embodiment 13, wherein said effective dosage is in a range of from 0.1 mg to 5.0 mg of said adenosine derivative once per week (Q1W).
15. The method of embodiment 14, wherein said effective dosage is in a range of from 0.1 mg to 0.75 mg of said adenosine derivative once per week (Q1W).
16. The method of any one of embodiments 9-15, wherein said pharmaceutical composition is formulated to produce an in vivo steady state C_{trough} of 4′-ethynyl-2-fluoro-2′-deoxyadenosine triphosphate (EFdA-TP) of 0.01 to 2 pmol/10⁶ human peripheral blood mononuclear cells (hPBMCs) for switching maintenance therapy.
17. The method of any one of embodiments 9-16, wherein said pharmaceutical composition is formulated to produce an in vivo steady state C_{trough} of 4′-ethynyl-2-fluoro-2′-deoxyadenosine triphosphate (EFdA-TP) of 0.01 to 0.5 pmol/10⁶ human peripheral blood mononuclear cells (hPBMCs) for treatment-naïve therapy.
18. The method of any one of embodiments 9-15, wherein said effective dosage is adjusted to produce an in vivo steady state C_{trough} of 4′-ethynyl-2-fluoro-2′-deoxyadenosine triphosphate (EFdA-TP) of 0.01 to 2 pmol/10⁶ human peripheral blood mononuclear cells (hPBMCs) for switching maintenance therapy.
19. The method of any one of embodiments 9-16, wherein said effective dosage is adjusted to produce an in vivo steady state C_{trough} of 4′-ethynyl-2-fluoro-2′-deoxyadenosine triphosphate (EFdA-TP) of 0.01 to 0.5 pmol/10⁶ human peripheral blood mononuclear cells (hPBMCs) for treatment-naïve therapy.
20. The method of any one of embodiments 9-19 further comprising the step of administering to said subject one or more anti-HIV agents selected from atazanavir, atazanavir sulfate, bictegravir, cabotegravir, dolutegravir, doravirine, efavirenz, tenofovir disoproxil fumarate, tenofovir alafenamide, elvitegravir, etravirine, darunavir, a combination of darunavir and cobicistat, rilpivirine, one or more capsid (CA) inhibitors, lenacapavir, and a combination thereof.
21. The method of any one of embodiments 9-20, wherein the HIV infection is caused by wild-type HIV-1, NRTI-resistant HIV-1, HIV-2, HIV having M184V mutations, HIV having K65R, or multidrug resistant HIV.
22. The method of any one of embodiments 9-21 further comprising:
   measuring purine nucleoside phosphorylase (PNP) activity prior to administering said pharmaceutical composition to produce baseline PNP activity and after administering said pharmaceutical composition to produce post admin PNP activity from samples of said subject;
   producing PNP activity data by comparing said post admin PNP activity to said baseline PNP activity; and
   adjusting said effective dosage based on said PNP activity data so said post admin PNP activity is in a range of from 20% to 200% of said baseline PNP activity.
23. A process for diagnosis and treatment or prevention of HIV infection in a subject in need thereof, said process comprising:
   measuring purine nucleoside phosphorylase (PNP) activity from a sample of said subject to produce individual baseline PNP activity of said subject, and
   optionally, measuring total lymphocyte counts and total CD4+ T-cell counts from a sample of the subject to produce individual baseline diagnostic data of the subject.
24. The process of embodiment 23 further comprising:
   comparing said individual baseline PNP activity to pre-stored normalized baseline PNP activity; and
   administering an effective dosage of a pharmaceutical composition to said subject if said individual baseline PNP activity is at least 20% or more of said pre-stored normalized baseline PNP activity.
25. The process of embodiment 24 further comprising:
   comparing said individual baseline diagnostic data to pre-stored normalized baseline diagnostic data; and
   administering an effective dosage of said pharmaceutical composition to said subject if said individual baseline diagnostic data is at least 20% or more of said pre-stored normalized baseline diagnostic data.
26. The process of any one of embodiments 24-25, wherein said pharmaceutical composition comprises at least a nucleotide, a nucleotide derivative, an adenosine derivative, at least an integrase inhibitor, at least a nucleoside RT inhibitors (NRTI), at least a non-nucleoside reverse transcriptase inhibitor (NNRTI), at least a protease inhibitor, or a combination thereof.
27. The process of embodiment 26, wherein said pharmaceutical composition comprises said adenosine derivative.
28. The process of embodiment 27, wherein said adenosine derivative comprises formula (1):

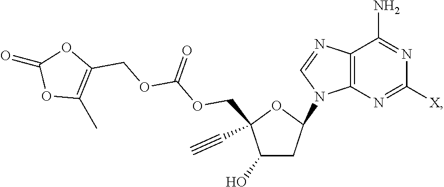

X is a halogen atom, 4′-ethynyl-2-fluoro-2′-deoxyadenosine (EFdA), a pharmaceutically acceptable salt, stereoisomer, tautomer, or solvate thereof, or a combination thereof.
29. The process of embodiment 28, wherein said adenosine derivative comprises formula (1-A):

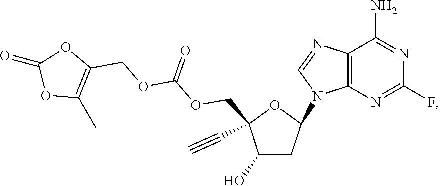 [SMILES: C#C[C@]1(COC(=O)OCc2oc(=O)oc2C)O[C@@H](n2cnc3c(N)nc(F)nc32)C[C@@H]1O]

a pharmaceutically acceptable salt, stereoisomer, tautomer, or solvate thereof, or a combination thereof.
30. The process of any one of embodiments 23-29, wherein each of said individual baseline PNP activity, said pre-stored normalized baseline PNP activity, or a combination thereof, is measured based on PNP enzymatic activity, PNP mRNA, PNP genomic DNA, PNP gene mutations, PNP gene single nucleotide polymorphisms (SNPs), or a combination thereof.
31. The process of any one of embodiments 24-30, wherein said individual baseline PNP activity is in a range of from 20% to 200% of said pre-stored normalized baseline PNP activity.
32. Use of an adenosine derivative and one or more pharmaceutically acceptable carriers for manufacturing a medicament for treating a disease, wherein said adenosine derivative is a compound of formula (1):

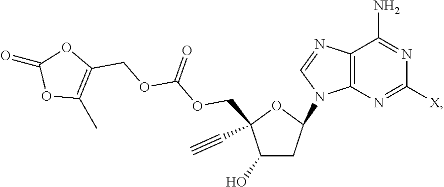

a pharmaceutically acceptable salt, stereoisomer, tautomer, or solvate thereof; wherein X is a halogen atom.
33. The use of embodiment 32, wherein X is F and said adenosine derivative is a compound of formula (1-A):

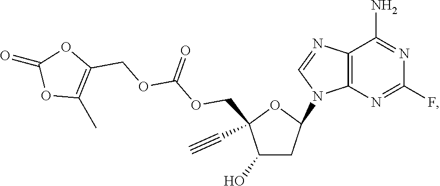 [SMILES: C#C[C@]1(COC(=O)OCc2oc(=O)oc2C)O[C@@H](n2cnc3c(N)nc(F)nc32)C[C@@H]1O]

a pharmaceutically acceptable salt, stereoisomer, tautomer, or solvate thereof.
34. The use of any one of embodiments 32-33, wherein said medicament is formulated as an implant, a controlled release implant, an oral suspension, an oral tablet, or an injectable composition suitable for intramuscular (IM), subcutaneous (SC) or parenteral injections.
35. The use of any one of embodiments 32-34, wherein said medicament is formulated to comprise in a range of from 0.1 mg to 10 mg of said adenosine derivative in a single dosage.
36. The use of embodiment 35, wherein said medicament comprises from 0.1 mg to 1.0 mg of said adenosine derivative.
37. The use of any one of embodiments 32-36, wherein said disease is an HIV infection.
38. The use of embodiment 37, wherein the HIV infection is caused by wild-type HIV-1, NRTI-resistant HIV-1, HIV-2, HIV having M184V mutations, HIV having K65R, or multidrug resistant HIV.

### EXAMPLES

The instant disclosure now will be exemplified by the following non-limiting examples.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Properties of the Adenosine Derivatives

Properties of the adenosine derivative are listed in Table 1 and Table 2. The adenosine derivative having the Formula 1-A was produced according to the process described in PCT publication WO2021021717, published on Feb. 4, 2021, herein incorporated by reference.

The prodrug adenosine derivative 1-A was administered to human subject. Plasma levels of the prodrug and the target drug EFdA were measured as described in aforementioned PCT publication WO2021021717.

The intracellular half-life of EFdA triphosphate (EFdA-TP) was measured as about 108 hours in human.

TABLE 1
Nomenclature and properties.

| Formula | | Molecular |
|---|---|---|
| ID | IUPAC Nomenclature | Weight |
| 1-A | ((2R,3S,5R)-5-(6-amino-2-fluoro-9H-purin- | 449.35 |
| | 9-yl)-2-ethynyl-3-hydroxytetrahydrofuran- | |
| | 2-yl)methyl ((5-methyl-2-oxo-1,3- | |
| | dioxol-4-yl)methyl) carbonate | |

TABLE 2
Conversion and Half Life Data.

| | Stability in Human Plasma | | Stability in Human Liver S9 | |
|---|---|---|---|---|
| | | Formation | | Formation |
| | | of EFdA | | of EFdA |
| Formula | Half-life | at 60 min | Half-life | at 60 min |
| 1-A | C | Yes | C | Yes |

Half-life ranges: A: >200 minutes; B: 50-200 minutes; C: <50 minutes.

Data showed that adenosine derivative 1-A can be converted to the target drug efficiently in human plasma and liver S9 assays.

### Purine Nucleoside Phosphorylase (PNP) Activity Assay

Human peripheral blood mononuclear cells (hPBMCs) samples from a person can be collected. Commercial fluorometric purine nucleoside phosphorylase activity assay kit can be used. Hypoxanthine formed from the breakdown of inosine can be measured with a PNP Probe. The fluorescent product can be measured at Ex/Em=535/587 nm to determine the PNP activity.

### Examples 1-17: Formulation for Tablets

Example 1: A pharmaceutical composition is formulated to contain 0.1 mg of the adenosine derivative having the Formula 1-A in one tablet.

Example 2: A pharmaceutical composition is formulated to contain 0.2 mg of the adenosine derivative having the Formula 1-A in one tablet.

Example 3: A pharmaceutical composition is formulated to contain 0.25 mg of the adenosine derivative having the Formula 1-A in one tablet.

Example 4: A pharmaceutical composition is formulated to contain 0.3 mg of the adenosine derivative having the Formula 1-A in one tablet.

Example 5: A pharmaceutical composition is formulated to contain 0.4 mg of the adenosine derivative having the Formula 1-A in one tablet.

Example 6: A pharmaceutical composition is formulated to contain 0.5 mg of the adenosine derivative having the Formula 1-A in one tablet.

Example 7: A pharmaceutical composition is formulated to contain 0.6 mg of the adenosine derivative having the Formula 1-A in one tablet.

Example 8: A pharmaceutical composition is formulated to contain 0.7 mg of the adenosine derivative having the Formula 1-A in one tablet.

Example 9: A pharmaceutical composition is formulated to contain 0.75 mg of the adenosine derivative having the Formula 1-A in one tablet.

Example 10: A pharmaceutical composition is formulated to contain 0.8 mg of the adenosine derivative having the Formula 1-A in one tablet.

Example 11: A pharmaceutical composition is formulated to contain 0.9 mg of the adenosine derivative having the Formula 1-A in one tablet.

Example 12: A pharmaceutical composition is formulated to contain 1.0 mg of the adenosine derivative having the Formula 1-A in one tablet.

Example 13: A pharmaceutical composition is formulated to contain 1.5 mg of the adenosine derivative having the Formula 1-A in one tablet.

Example 14: A pharmaceutical composition is formulated to contain 1.75 mg of the adenosine derivative having the Formula 1-A in one tablet.

Example 15: A pharmaceutical composition is formulated to contain 2.0 mg of the adenosine derivative having the Formula 1-A in one tablet.

Example 16: A pharmaceutical composition is formulated to contain 5.0 mg of the adenosine derivative having the Formula 1-A in one tablet.

Example 17: A pharmaceutical composition is formulated to contain 10.0 mg of the adenosine derivative having the Formula 1-A in one tablet.

### Examples 18-45: Formulation for Injection Solutions

Example 18: A pharmaceutical composition is formulated to contain 0.1 mg of the adenosine derivative having the Formula 1-A in one vial as lyophilized dry powder, that can be reconstituted with sterile saline having a volume in a range of from 0.1 mL to 1.0 mL.

Example 19: A pharmaceutical composition is formulated to contain 0.25 mg of the adenosine derivative having the Formula 1-A in one vial as lyophilized dry powder, that can be reconstituted with sterile saline having a volume in a range of from 0.1 mL to 1.0 mL.

Example 20: A pharmaceutical composition is formulated to contain 0.5 mg of the adenosine derivative having the Formula 1-A in one vial as lyophilized dry powder, that can be reconstituted with sterile saline having a volume in a range of from 0.1 mL to 1.0 mL.

Example 21: A pharmaceutical composition is formulated to contain 0.7 mg of the adenosine derivative having the Formula 1-A in one vial as lyophilized dry powder, that can be reconstituted with sterile saline having a volume in a range of from 0.1 mL to 1.0 mL.

Example 22: A pharmaceutical composition is formulated to contain 0.75 mg of the adenosine derivative having the Formula 1-A in one vial as lyophilized dry powder, that can be reconstituted with sterile saline having a volume in a range of from 0.1 mL to 1.0 mL.

Example 23: A pharmaceutical composition is formulated to contain 0.8 mg of the adenosine derivative having the Formula 1-A in one vial as lyophilized dry powder, that can be reconstituted with sterile saline having a volume in a range of from 0.1 mL to 1.0 mL.

Example 24: A pharmaceutical composition is formulated to contain 0.9 mg of the adenosine derivative having the Formula 1-A in one vial as lyophilized dry powder, that can be reconstituted with sterile saline having a volume in a range of from 0.1 mL to 1.0 mL.

Example 25: A pharmaceutical composition is formulated to contain 1.0 mg of the adenosine derivative having the Formula 1-A in one vial as lyophilized dry powder, that can be reconstituted with sterile saline having a volume in a range of from 0.1 mL to 1.0 mL.

Example 26: A pharmaceutical composition is formulated to contain 1.25 mg of the adenosine derivative having the Formula 1-A in one vial as lyophilized dry powder, that can be reconstituted with sterile saline having a volume in a range of from 0.1 mL to 1.0 mL.

Example 27: A pharmaceutical composition is formulated to contain 1.5 mg of the adenosine derivative having the Formula 1-A in one vial as lyophilized dry powder, that can be reconstituted with sterile saline having a volume in a range of from 0.1 mL to 1.0 mL.

Example 28: A pharmaceutical composition is formulated to contain 1.75 mg of the adenosine derivative having the Formula 1-A in one vial as lyophilized dry powder, that can be reconstituted with sterile saline having a volume in a range of from 0.1 mL to 1.0 mL.

Example 29: A pharmaceutical composition is formulated to contain 1.8 mg of the adenosine derivative having the Formula 1-A in one vial as lyophilized dry powder, that can be reconstituted with sterile saline having a volume in a range of from 0.1 mL to 1.0 mL.

Example 30: A pharmaceutical composition is formulated to contain 1.9 mg of the adenosine derivative having the Formula 1-A in one vial as lyophilized dry powder, that can be reconstituted with sterile saline having a volume in a range of from 0.1 mL to 1.0 mL.

Example 31: A pharmaceutical composition is formulated to contain 2.0 mg of the adenosine derivative having the Formula 1-A in one vial as lyophilized dry powder, that can be reconstituted with sterile saline having a volume in a range of from 0.1 mL to 1.0 mL.

Example 32: A pharmaceutical composition is formulated to contain 2.5 mg of the adenosine derivative having the Formula 1-A in one vial as lyophilized dry powder, that can be reconstituted with sterile saline having a volume in a range of from 0.1 mL to 1.0 mL.

Example 33: A pharmaceutical composition is formulated to contain 5.0 mg of the adenosine derivative having the Formula 1-A in one vial as lyophilized dry powder, that can be reconstituted with sterile saline having a volume in a range of from 0.1 mL to 1.0 mL.

Example 34: A pharmaceutical composition is formulated to contain 10 mg of the adenosine derivative having the Formula 1-A in one vial as lyophilized dry powder, that can be reconstituted with sterile saline having a volume in a range of from 0.1 mL to 1.0 mL.

Examples 35-51: Each of the formulations in Examples 18-34 are produced as liquid and stored at 4° C.

### Example 52: Formulation for In Situ Implant

The implant formulations were prepared to achieve 5% (Prodrug Formulation A) or 10% (Prodrug Formulation B) drug load by weight.

About 250 mg of each of the Prodrug Formulations was added to 5 mL of PBS. A gel was formed instantly with each of the Prodrug Formulations demonstrating in-situ gel formation that is suitable for in situ implant.

### Example 53: Safety, Tolerability, and Pharmacokinetics of Adenosine Derivative Having Formula 1-A (“1-A”)

This example describes results from a Phase I, randomized, double-blind, placebo-controlled study for adenosine derivative having Formula 1-A (“1-A”), which is a prodrug of islatravir (ISL). Following oral absorption, 1-A is rapidly converted to ISL, which is metabolized intracellularly to the active metabolite, ISL triphosphate (ISL-TP). The objectives of the study were to assess the safety, tolerability, and PK profiles of 1-A and ISL in plasma; and ISL diphosphate (ISL-DP) and ISL-TP in human PBMCs.

Methods. Healthy adult subjects were enrolled in five single ascending dose and two multiple ascending dose cohorts. 1-A was administered to single dose cohorts in amounts of 10 mg, 25 mg, 50 mg, 100 mg or 200 mg. 1-A was administered to multiple dose cohorts in amounts of 10 mg per week for 3 weeks or 25 mg per week for 3 weeks. 1-A was orally administered in the fasted state using an oral solution formulation. Safety-related assessments included physical examinations, ECGs, vital signs, AEs, and standard clinical laboratory tests.

Results. After single or multiple oral administrations of 1-A up to 200 mg, no measurable systemic exposure to 1-A was detected (LLOQ of 1.0 ng/mL), suggesting fast and efficient release of EFdA from 1-A. Dose-dependent ISL plasma PK profiles were observed after single or multiple oral doses of 1-A over the range of 10 mg to 200 mg. No meaningful EFdA accumulation was observed in plasma. Efficient intracellular formation of ISL-TP in PBMCs was observed in a dose-dependent manner. Consistent with the long cellular half-lives, significant ISL-TP accumulations were observed after 3 weekly doses of 25 mg 1-A. Tables 3 and 4 provide summary pharmacokinetics from subjects that received 3 weekly doses of 25 mg 1-A.

TABLE 3
Summary Pharmacokinetics of Islatravir in Plasma Following
3 Weekly Oral Doses of 25 mg 1-A to Healthy Adult Subjects.

| | | | | | Apparent |
|---|---|---|---|---|---|
| | AUCₗₐₛₜ | Cₘₐₓ | C_{168 h} | Tₘₐₓ | terminal t_{1/2} |
| | (h*ng/mL) | (ng/mL) | (ng/mL) | (h) | (h) |
| Day 1 | 511 | 120 | NC | 0.53 | 45 |
| (N = 6) | (21.7) | (18.8) | | (0.5, 1.0) | (33, 52) |
| Day 15 | 620 | 121 | 1.10 | 0.5 | 76 |
| (N = 6) | (22.3) | (26.0) | (12.9) | (0.5, 0.52) | (59, 91) |

Note
Data reported in mean (% CV) except Tₘₐₓ and apparent terminal t_{1/2} in median (Q1, Q3).

TABLE 4
Summary Pharmacokinetics of ISL-TP in PBMCs Following 3
Weekly Oral Doses of 25 mg 1-A to Healthy Adult Subjects.

| | AUCₗₐₛₜ | Cₘₐₓ | C_{168 h} | | Apparent |
|---|---|---|---|---|---|
| | (h*pmol/10⁶ | (pmol/10⁶ | (pmol/10⁶ | Tₘₐₓ | terminal t_{1/2} |
| | cells) | cells) | cells) | (h) | (h) |
| Day 1 | 617 | 5.70 | 2.70 | 24 | NC |
| (N = 6) | (20.8) | (23.8) | (29.5) | (12, 24) | |
| Day 15 | 2210 | 15.4 | 4.70 | 7.0 | 177 |
| (N = 6) | (21.3) | (16.5) | (27.6) | (7.0, 12) | (172, 183) |

Note
Data reported in mean (% CV) except Tₘₐₓ and apparent terminal t_{1/2}, in median (Q1, Q3).

Treatment-emergent adverse events (TEAEs) from cohorts receiving 3 weekly doses of 1-A are summarized in Table 5.

TABLE 5
Overall Summary of Subjects with Treatment-emergent Adverse
Events Following 3 Weekly Oral Doses of 10 mg and 25 mg 1-A.

| | 1-A | 1-A | Pooled |
|---|---|---|---|
| Number (%) of | 10 mg QW × 3 | 25 mg QW × 3 | Placebo |
| Subjects with Any | (N = 3) | (N = 6) | (N = 3) |
| TEAE | 2 (66.7%) | 2 (33.3%) | 3 (100%) |
| Grade 1 | 2 (66.7%) | 2 (33.3%) | 3 (100%) |
| Grade 2, 3, 4, or 5 | 0 | 0 | 0 |
| TEAE Related to | 2 (66.7%) | 2 (33.3%) | 2 (66.7%) |
| Study Drug | | | |
| Grade 1 | 2 (66.7%) | 2 (33.3%) | 2 (66.7%) |
| Grade 2, 3, 4, or 5 | 0 | 0 | 0 |
| Treatment-emergent | 0 | 0 | 0 |
| Serious AE | | | |
| TEAE Leading to | 0 | 0 | 0 |
| Premature Discontinuation | | | |
| of Study Drug | | | |
| Death | 0 | 0 | 0 |

Conclusions. 1-A was generally well tolerated at single doses up to 200 mg and repeat doses up to 25 mg. PK profiles including ISL in plasma and ISL-TP in PBMC cells after single and multiple oral administrations of 1-A demonstrated linear pharmacokinetics and achieved therapeutic targets.

1. A method of treating an HIV infection in a subject comprising orally administering to the subject a once-weekly dose of from about 0.1 mg to about 25 mg of an EFdA prodrug or a pharmaceutically acceptable salt thereof for a plurality of weeks.

2. The method of claim 1, wherein the EFdA prodrug has the structure:

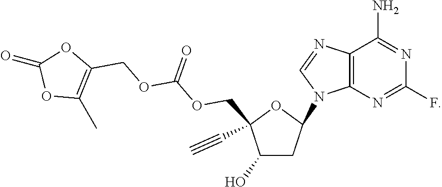 [SMILES: C#C[C@]1(COC(=O)OCc2oc(=O)oc2C)O[C@@H](n2cnc3c(N)nc(F)nc32)C[C@@H]1O]

3. (canceled)

4. The method of claim 2, wherein the once-weekly dose is from about 1 mg to about 10 mg of the EFdA prodrug or pharmaceutically acceptable salt.

5. The method of claim 4, wherein the once-weekly dose is from about 1 mg to about 2 mg of the EFdA prodrug or pharmaceutically acceptable salt.

6. The method of claim 5, wherein the once-weekly dose is about 1.25 mg of the EFdA prodrug or pharmaceutically acceptable salt.

7. The method of claim 1, wherein the once-weekly dose is administered as a tablet comprising the EFdA prodrug or pharmaceutically acceptable salt.

8. A method of treating an HIV infection in a subject comprising orally administering to the subject a once-daily dose of from about 0.1 mg to about 5 mg of an EFdA prodrug or a pharmaceutically acceptable salt thereof for a plurality of weeks.

9. The method of claim 8, wherein the EFdA prodrug has the structure:

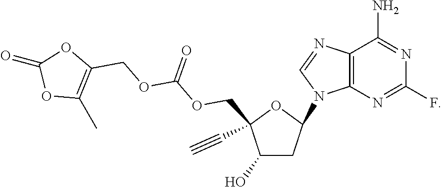 [SMILES: C#C[C@]1(COC(=O)OCc2oc(=O)oc2C)O[C@@H](n2cnc3c(N)nc(F)nc32)C[C@@H]1O]

10. (canceled)

11. The method of claim 9, wherein the once-daily dose is from about 0.1 mg to about 0.75 mg of the EFdA prodrug or pharmaceutically acceptable salt.

12. The method of claim 11, wherein the once-daily dose is about 0.25 mg or 0.38 mg of the EFdA prodrug or pharmaceutically acceptable salt.

13. The method of claim 8, wherein the once-daily dose is administered as a tablet comprising the EFdA prodrug or pharmaceutically acceptable salt.

14. The method of claim 1, wherein the HIV infection is caused by wild-type HIV-1, NRTI-resistant HIV-1, HIV-2, HIV having a M184V mutation, HIV having K65R, or multidrug resistant HIV.

15. The method of claim 1, wherein the HIV infection is an HIV-1 infection.

16. The method of claim 1, wherein the subject has reduced expression of:
(a) deoxycytidine kinase;
(b) adenosine deaminase; and/or
(c) purine nucleoside phosphorylase (PNP) compared to a treatment-naïve subject, compared to a treatment-naïve subject.

17. The method of claim 16, wherein the subject is at risk for CD4⁺ lymphocyte count reduction compared to a treatment-naïve subject.

18. The method of claim 1, further comprising administering to the subject one or more anti-HIV agents selected from atazanavir, atazanavir sulfate, bictegravir, cabotegravir, dolutegravir, doravirine, efavirenz, tenofovir disoproxil fumarate, tenofovir alafenamide, elvitegravir, etravirine, darunavir, a combination of darunavir and cobicistat, rilpivirine, lenacapavir, and a combination thereof.

19.-37. (canceled)

38. The method of claim 8, wherein the HIV infection is caused by wild-type HIV-1, NRTI-resistant HIV-1, HIV-2, HIV having a M184V mutation, HIV having K65R, or multidrug resistant HIV.

39. The method of claim 8, wherein the HIV infection is an HIV-1 infection.

40. The method of claim 8, wherein the subject has reduced expression of:
(a) deoxycytidine kinase;
(b) adenosine deaminase; and/or
(c) purine nucleoside phosphorylase (PNP) compared to a treatment-naïve subject, compared to a treatment-naïve subject.

41. The method of claim 40, wherein the subject is at risk for CD4⁺ lymphocyte count reduction compared to a treatment-naïve subject.

42. The method of claim 8, further comprising administering to the subject one or more anti-HIV agents selected from atazanavir, atazanavir sulfate, bictegravir, cabotegravir, dolutegravir, doravirine, efavirenz, tenofovir disoproxil fumarate, tenofovir alafenamide, elvitegravir, etravirine, darunavir, a combination of darunavir and cobicistat, rilpivirine, lenacapavir, and a combination thereof.

43. The method of claim 18, wherein the EFdA prodrug or pharmaceutically acceptable salt thereof and the one or more anti-HIV agents are administered simultaneously.

44. The method of claim 18, wherein the EFdA prodrug or pharmaceutically acceptable salt thereof and the one or more anti-HIV agents are administered sequentially.

45. The method of claim 42, wherein the EFdA prodrug or pharmaceutically acceptable salt thereof and the one or more anti-HIV agents are administered simultaneously.

46. The method of claim 42, wherein the EFdA prodrug or pharmaceutically acceptable salt thereof and the one or more anti-HIV agents are administered sequentially.
